(19) United States
(12) Patent Application Publication
(10) Pub. No.: US 2024/0207407 A1
(43) Pub. Date: Jun. 27, 2024

(54) EGFRVIII BINDING PROTEINS

(71) Applicants: The Walter and Eliza Hall Institute of Medical Research, Parkville (AU); Myrio Therapeutics Pty Ltd, Blackbum North (AU)

(72) Inventors: Misty JENKINS, Parkville (AU); Rebecca ABBOTT, Parkville (AU); Ryan CROSS, Parkville (AU); Fiona Margaret GRACEY, Blackburn North (AU); Rebecca Elizabeth WOOD, Blackburn North (AU); Benjamin Ross KIEFEL, Blackburn North (AU); Matthew David BEASLEY, Blackburn North (AU)

(21) Appl. No.: 18/288,475

(22) PCT Filed: Apr. 26, 2022
(86) PCT No.: PCT/AU2022/050379

§ 371 Date: Oct. 26, 2023

(30) Foreign Application Priority Data
Apr. 26, 2021 (AU) 2021901226
Apr. 26, 2021 (AU) 2021901227

(51) Int. Cl. A61K 39/00; A61P 35/00; C07K 14/705; C07K 14/725; C07K 14/735; C07K 16/28; C12N 15/86; G01N 33/574

(52) U.S. Cl. CPC A61K 39/464404; A61K 39/4611; A61K 39/4613; A61K 39/4631; A61K 39/4633; A61P 35/00; C07K 14/70503; C07K 14/7051; C07K 14/70517; C07K 14/70521; C07K 14/70535; C07K 16/2863; C12N 15/86; G01N 33/57492; A61K 39/00; C07K 2317/31; C07K 2317/622; C07K 2319/02; C07K 2319/03; C07K 2319/30; C12N 2740/15043; C12N 2800/22

(57) ABSTRACT

The present invention relates generally to proteins which bind to human epidermal growth factor receptor variant III (EGFRvIII). The present invention also relates to chimeric antigen receptors (CARs) comprising the EGFRvIII binding proteins, nucleic acids and vectors encoding the CARs, as well as cells comprising the CARs. The present invention also relates to methods of treating and diagnosing diseases such as cancer.

The present application claims priority from AU2021901227, filed 26 Apr. 2021 and AU2021901226, filed 26 Apr. 2021, the entire contents of each of which are incorporated herein by reference.

### FIELD OF THE INVENTION

The present invention relates generally to proteins which bind to human epidermal growth factor receptor variant III (EGFRvIII). The present invention also relates to chimeric antigen receptors (CARs) comprising the EGFRvIII binding proteins, nucleic acids and vectors encoding the CARs, as well as cells comprising the CARs. The present invention also relates to methods of treating and diagnosing diseases such as cancer.

### BACKGROUND OF THE INVENTION

EGFRvIII is the most common mutant variant of EGFR observed in human tumours, and can be detected in ˜30% of newly diagnosed glioblastoma multiforme (GBM) cases. GBM is the most aggressive and malignant adult brain tumour, with 5-year expected survival rates of 5%. The current treatments of surgery, chemotherapy and radiotherapy have not improved survival in decades.

EGFRvIII mutations have also been shown to be present in murine fibroblasts and in breast, ovarian, colon, lung and prostate cancers cells.

The mutation present in EGFRvIII is an in-frame deletion of 801 base pairs from exons 2 to 7, that splits a codon and creates a glycine residue at the junction, resulting in a tumour specific and immunogenic neoepitope, and rendering the protein constitutively active. This mutation also confers resistance to radiation and chemotherapy, giving patients with the mutation an even poorer prognostic outcome.

There is therefore a need for new EGFRvIII binding molecules that can be used in cancer therapy.

### SUMMARY OF THE INVENTION

The present inventors have identified binding proteins which bind to EGFRvIII with high affinity. These binding proteins can be used both in isolation (e.g., as isolated antibodies or fragments thereof) or within chimeric antigen receptor (CAR) constructs for CAR-T cell therapy, for example.

Thus, in an aspect, the present invention provides a binding protein comprising an antigen binding site which binds to human epidermal growth factor receptor variant III (EGFRvIII) with an affinity of at least about 1 nM. In some embodiments, the binding protein binds to EGFRvIII with an affinity of at least about 0.75 nM, at least about 0.5 nM, at least about 0.25 nM, at least about 0.1 nM, at least about 0.075 nM, at least about 0.05 nM, at least about 0.025 nM, or at least about 0.01 nM.

In some embodiments, the affinity is determined in an assay in which the binding protein is immobilized and soluble EGFRvIII is contacted with the binding protein. In an embodiment, the soluble EGFRvIII is an extracellular domain of EGFRvIII (e.g., an EGFRvIII construct from which the transmembrane and intracellular domains have been removed).

In an embodiment, the affinity of the binding protein for EGFRvIII is at least 20-fold, at least 100-fold, or at least 1000-fold stronger than its affinity for wild-type epidermal growth factor receptor (EGFR). In an embodiment, the binding protein does not detectably bind to EGFR in an assay in which the binding protein is immobilized and soluble EGFR is contacted with the binding protein. In an embodiment, the affinity of the binding protein for EGFRvIII is at least 20-fold, at least 40-fold, at least 60-fold, at least 80-fold, at least 100-fold, at least 200-fold, at least 300-fold, at least 400-fold, at least 500-fold, at least 600-fold, at least 800-fold, at least 1000-fold, at least 5000-fold, or at least 10000-fold stronger than its affinity for wild-type epidermal growth factor receptor (EGFR). Advantageously, in such embodiments the binding proteins have greater tumour specificity as a result of their lower affinity for EGFR expressed on non-cancerous cells.

The present inventors have found that, in some embodiments, the binding proteins are also capable of binding to EGFR when overexpressed on the surface of cancer cells. Thus, in some embodiments, the binding protein binds to EGFR on cancer cells that overexpress EGFR. In some embodiments, the binding protein binds to EGFR on cancer cells that overexpress EGFR due to EGFR gene amplification. In an embodiment, the binding protein binds to an epitope exposed on cells expressing abnormal or overexpressed EGFR (e.g., cancer cells), but not exposed on wild type cells. In an embodiment, the binding protein binds to EGFR which is present on cancer cells but which is not detectable on wild-type cells. Advantageously, in these embodiments the binding protein can be used in a therapy against cancers that overexpress EGFR (e.g., as a result of gene amplification) as well as cancers that express the EGFRvIII variant, thereby expanding the potential therapeutic indications for these binding proteins.

In some embodiments, the binding protein binds to EGFR on gliobastoma cells or epithelial cells. In an embodiment, the affinity of the binding protein for the EGFR overexpressed by the cancer cells is at least 20-fold, at least 100-fold, or at least 1000-fold stronger than its affinity for EGFR expressed by wild-type cells, or the binding protein does not detectably bind to EGFR expressed by wild-type cells. Affinity can be assessed by assays preformed with cell lines such as U87, U251 or A431.

In some embodiments, the binding protein binds to the same epitope in EGFRvIII as that bound by a protein comprising a heavy chain variable region (V_{H}) comprising an amino acid sequence set forth in SEQ ID NO:3 and a light chain variable region (V_{L}) comprising an amino acid sequence set forth in SEQ ID NO:4.

In an aspect of the invention, there is provided a human epidermal growth factor receptor variant III (EGFRvIII) binding protein comprising an antigen binding domain which binds to EGFRvIII, wherein the binding protein competitively inhibits binding of EGFRvIII to an antibody comprising:
a heavy chain variable region (V_{H}) comprising a CDR1, a CDR2, and a CDR3 comprising the sequences set forth in SEQ ID NOs: 13, 14, and 15 respectively; and
a light chain variable region (V_{L}) comprising a CDR1, a CDR2, and a CDR3 comprising the sequences set forth in SEQ ID NOs: 16, 17, and 18 respectively; or
a heavy chain variable region (V_{H}) comprising a CDR1, a CDR2, and a CDR3 comprising the sequences set forth in SEQ ID NOs: 19, 20, and 21 respectively; and
a light chain variable region (V_{L}) comprising a CDR1, a CDR2, and a CDR3 comprising the sequences set forth in SEQ ID NOs: 22, 23, and 24 respectively.

In an embodiment, the binding protein competitively inhibits binding of EGFRvIII to an antibody comprising:
a V_{H} comprising an amino acid sequence set forth in SEQ ID NO:3 and a V_{L} comprising an amino acid sequence set forth in SEQ ID NO:4; or
a V_{H} comprising an amino acid sequence set forth in SEQ ID NO:5 and a V_{L} comprising an amino acid sequence set forth in SEQ ID NO:6.

In an embodiment, the EGFRvIII binding protein binds to human EGFRvIII with an affinity of at least about 1 nM.

In an embodiment, the binding protein comprises:
a heavy chain variable region (V_{H}) comprising a CDR1, a CDR2, and a CDR3 comprising the sequences set forth in SEQ ID NOs: 7, 8, and 9 respectively; and/or
a light chain variable region (V_{L}) comprising a CDR1, a CDR2, and a CDR3 comprising the sequences set forth in SEQ ID NOs: 10, 11, and 12 respectively; or
a heavy chain variable region (V_{H}) comprising a CDR1, a CDR2, and a CDR3 comprising the sequences set forth in SEQ ID NOs: 13, 14, and 15 respectively; and/or
a light chain variable region (V_{L}) comprising a CDR1, a CDR2, and a CDR3 comprising the sequences set forth in SEQ ID NOs: 16, 17, and 18 respectively.

In an embodiment, the binding protein comprises:
a heavy chain variable region (V_{H}) comprising a CDR1 comprising an amino acid sequence having no more than 2 amino acid substitutions relative to SEQ ID NO: 13, a CDR2 comprising an amino acid sequence having no more than 4 amino acid substitutions relative to SEQ ID NO: 14, and a CDR3 comprising an amino acid sequence having no more than 3 amino acid substitutions relative to SEQ ID NO: 15, and/or
a light chain variable region (V_{L}) comprising a CDR1 comprising an amino acid sequence having no more than 3 amino acid substitutions relative to SEQ ID NO:16, a CDR2 comprising an amino acid sequence having no more than 2 amino acid substitutions relative to SEQ ID NO: 17, and a CDR3 comprising an amino acid sequence having no more than 3 amino acid substitutions relative to SEQ ID NO: 18; or
a heavy chain variable region (V_{H}) comprising a CDR1 comprising an amino acid sequence having no more than 2 amino acid substitutions relative to SEQ ID NO:19, a CDR2 comprising an amino acid sequence having no more than 4 amino acid substitutions relative to SEQ ID NO:20, and a CDR3 comprising an amino acid sequence having no more than 3 amino acid substitutions relative to SEQ ID NO:21, and/or
a light chain variable region (V_{L}) comprising a CDR1 comprising an amino acid sequence having no more than 3 amino acid substitutions relative to SEQ ID NO:22, a CDR2 comprising an amino acid sequence having no more than 2 amino acid substitutions relative to SEQ ID NO:23, and a CDR3 comprising an amino acid sequence having no more than 3 amino acid substitutions relative to SEQ ID NO:24.

In an embodiment, a CDR sequence referred to above has no more than 4, no more than 3, no more than 2, or no more than 1 amino acid substitution(s) relative to the recited SEQ ID NO.

In an embodiment, the binding protein comprises:
a heavy chain variable region (V_{H}) comprising a CDR1, a CDR2, and a CDR3 comprising the sequences set forth in SEQ ID NOs: 13, 14, and 15 respectively; and/or
a light chain variable region (V_{L}) comprising a CDR1, a CDR2, and a CDR3 comprising the sequences set forth in SEQ ID NOs: 16, 17, and 18 respectively; or
a heavy chain variable region (V_{H}) comprising a CDR1, a CDR2, and a CDR3 comprising the sequences set forth in SEQ ID NOs: 19, 20, and 21 respectively; and/or
a light chain variable region (V_{L}) comprising a CDR1, a CDR2, and a CDR3 comprising the sequences set forth in SEQ ID NOs: 22, 23, and 24 respectively.

In an emboidment, the binding protein comprises:
a V_{H} comprising a CDR1, CDR2, and a CDR3 comprising the amino acid sequences set forth in SEQ ID NOs: 19, 20, and 21 respectively, and
a V_{L} comprising a CDR1, CDR2, and a CDR3 comprising the amino acid sequences set forth in SEQ ID NOs: 19, 20, and 21 respectively.

In an embodiment, the binding protein comprises:
a heavy chain variable region (V_{H}) comprising an amino acid sequence at least about 70% identical, at least about 80% identical, at least about 90% identical, or at least about 95% identical to the sequence set forth in SEQ ID NO:3, and/or
a light chain variable region (V_{L}) comprising an amino acid sequence at least about 70% identical, at least about 80% identical, at least about 90% identical, or at least about 95% identical to the sequence set forth in SEQ ID NO:4; or
a heavy chain variable region (V_{H}) comprising an amino acid sequence at least about 70% identical, at least about 80% identical, at least about 90% identical, or at least about 95% identical to the sequence set forth in SEQ ID NO:5, and/or
a light chain variable region (V_{L}) comprising an amino acid sequence at least about 70% identical, at least about 80% identical, at least about 90% identical, or at least about 95% identical to the sequence set forth in SEQ ID NO:6.

In an embodiment, the binding protein comprises:
a heavy chain variable region (V_{H}) comprising an amino acid sequence set forth in SEQ ID NO:3 and/or a light chain variable region (V_{L}) comprising an amino acid sequence set forth in SEQ ID NO:4; or
a heavy chain variable region (V_{H}) comprising an amino acid sequence set forth in SEQ ID NO:5 and/or a light chain variable region (V_{L}) comprising an amino acid sequence set forth in SEQ ID NO:6.

In an embodiment, the binding protein comprises a V_{H} comprising the amino acid sequence set forth in SEQ ID NO:3 and a V_{L} comprising the amino acid sequence set forth in SEQ ID NO:4.

In an embodiment, the binding protein comprises a V_{H} comprising the amino acid sequence set forth in SEQ ID NO:5 and a V_{L} comprising the amino acid sequence set forth in SEQ ID NO:6.

In some embodiments, the binding protein comprises:
(i) a V_{H} comprising a CDR1 comprising an amino acid sequence having no more than 2 amino acid substitutions relative to SEQ ID NO:41, a CDR2 comprising an amino acid sequence having no more than 4 amino acid substitutions relative to SEQ ID NO:42, and a CDR3 comprising an amino acid sequence having no more than 3 amino acid substitutions relative to SEQ ID NO:43, and/or
(ii) a V_{L} comprising a CDR1 comprising an amino acid sequence having no more than 3 amino acid substitutions relative to SEQ ID NO:44, a CDR2 comprising an amino acid sequence having no more than 2 amino acid substitutions relative to SEQ ID NO:45, and a CDR3 comprising an amino acid sequence having no more than 3 amino acid substitutions relative to SEQ ID NO:46. In an embodiment, a CDR sequence referred to above has no more than 4, no more than 3, no more than 2, or no more than 1 amino acid substitution(s) relative to the recited SEQ ID NO.

In some embodiments, the binding protein comprises:
(i) a V_{H} comprising a CDR1, CDR2, and a CDR3 comprising the amino acid sequences set forth in SEQ ID NOs: 41, 42, and 43 respectively, and/or
(ii) a V_{L} comprising a CDR1, CDR2, and a CDR3 comprising the amino acid sequences set forth in SEQ ID NOs: 44, 45, and 46 respectively.

In some embodiments, the binding protein comprises:
(i) a V_{H} comprising a CDR1, CDR2, and a CDR3 comprising the amino acid sequences set forth in SEQ ID NOs: 41, 42, and 43 respectively, and
(ii) a V_{L} comprising a CDR1, CDR2, and a CDR3 comprising the amino acid sequences set forth in SEQ ID NOs: 44, 45, and 46 respectively.

In some embodiments, the binding protein comprises:
(i) a V_{H} comprising a CDR1 comprising an amino acid sequence having no more than 2 amino acid substitutions relative to SEQ ID NO:47, a CDR2 comprising an amino acid sequence having no more than 4 amino acid substitutions relative to SEQ ID NO:48, and a CDR3 comprising an amino acid sequence having no more than 3 amino acid substitutions relative to SEQ ID NO:49, and/or
(ii) a V_{L} comprising a CDR1 comprising an amino acid sequence having no more than 3 amino acid substitutions relative to SEQ ID NO:50, a CDR2 comprising an amino acid sequence having no more than 2 amino acid substitutions relative to SEQ ID NO:51, and a CDR3 comprising an amino acid sequence having no more than 3 amino acid substitutions relative to SEQ ID NO:52.

In an embodiment, a CDR sequence referred to above has no more than 4, no more than 3, no more than 2, or no more than 1 amino acid substitution(s) relative to the recited SEQ ID NO.

In an embodiment, a CDR sequence referred to above has no more than 4, no more than 3, no more than 2, or no more than 1 amino acid substitution(s) relative to the recited SEQ ID NO.

In some embodiments, the binding protein comprises:
(i) a V_{H} comprising a CDR1, CDR2, and a CDR3 comprising the amino acid sequences set forth in SEQ ID NOs: 47, 48, and 49 respectively, and/or
(ii) a V_{L} comprising a CDR1, CDR2, and a CDR3 comprising the amino acid sequences set forth in SEQ ID NOs: 50, 51, and 52 respectively.

In some embodiments, the binding protein comprises:
(i) a V_{H} comprising a CDR1, CDR2, and a CDR3 comprising the amino acid sequences set forth in SEQ ID NOs: 47, 48, and 49 respectively, and
(ii) a V_{L} comprising a CDR1, CDR2, and a CDR3 comprising the amino acid sequences set forth in SEQ ID NOs: 50, 51, and 52 respectively.

In some embodiments, the binding protein comprises a V_{H} and a V_{L}, wherein the V_{H} and V_{L} bind to form a Fv comprising the antigen binding site.

In some embodiments, the V_{H} and the V_{L} are in a single polypeptide chain.

In some embodiments, the binding protein comprises:
(i) a single chain Fv fragment (scFv);
(ii) a dimeric scFv (di-scFv); or
(iii) at least one of (i) and/or (ii) linked to a constant region of an antibody, a fragment crystallizable (Fc) region or a heavy chain constant domain (C_{H}) 2 and/or C_{H}3.

In some embodiments, the binding protein comprises a scFv.

In some embodiments, the binding protein comprises an amino acid sequence at least about 70% identical, at least about 80% identical, at least about 90% identical, or at least about 95% identical to the sequence set forth in SEQ ID NO:27.

In some embodiments, the binding protein comprises the amino acid sequence set forth in SEQ ID NO:27.

In some embodiments, the binding protein is a bi-specific T cell engager (BiTE).

In some embodiments, the V_{L} and V_{H} are in separate polypeptide chains.

In some embodiments, the binding protein comprises:
(i) a diabody;
(ii) a triabody;
(iii) a tetrabody;
(iv) a Fab;
(v) a F(ab′)₂;
(vi) a Fv; or
(vii) at least one of (i) to (vi) linked to a constant region of an antibody, an Fc region or a C_{H}2 and/or C_{H}3.

In some embodiments. the binding protein comprises a Fc region. In an embodiment. the binding protein is an antibody.

In some embodiments, the binding protein is conjugated to another compound. In some embodiments, the binding protein is conjugated to a half-life extending moiety. In some embodiments, the binding protein is conjugated to a polymer (e.g., PEG). In some embodiments, the binding protein is conjugated to a cytotoxic agent.

In some embodiments, the binding protein is chimeric, de-immunized, humanized, human or primatized. In some embodiments, the binding protein is a human protein.

In another aspect, the present invention provides a composition comprising the binding protein of the invention and a pharmaceutically acceptable carrier.

In another aspect, the present invention provides a cell comprising the binding protein of the invention.

Advantageously, the present inventors have found that the binding proteins of the invention can be incorporated into chimeric antigen receptors (CARs) to produce CAR expressing cells for targeting EGFRvIII-expressing cells. Thus, in another aspect, the present invention provides a CAR comprising an antigen-binding domain of the invention, a transmembrane domain, and an intracellular domain.

In some embodiments, the intracellular domain comprises a primary signalling domain and a costimulatory domain. In an embodiment, the intracellular domain comprises two or more costimulatory domains. In an alternative embodiment, the CAR does not comprise a costimulatory domain.

In some embodiments, the primary signalling domain comprises a CD3 zeta, CD3 gamma, CD3 delta, CD3 epsilon, common FcR gamma (FCER1G), FcR beta (Fc Epsilon Rib), CD79a, CD79b, Fcgamma Rlla, DAP10, or DAP12 primary signalling domain.

In some embodiments, the primary signalling domain is a CD3 zeta primary signalling domain. In some embodiments, the primary signalling domain comprises an amino acid sequence which is at least about 70% identical, at least about 80% identical, at least about 90% identical, at least about 95% or about 100% identical to the sequence set forth in SEQ ID NO:35.

In some embodiments, the costimulatory domain comprises a CD28, 4-1BB (CD137), OX40, CD27, CD30, CD40, CD134, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFl), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD18, LFA-1, ITGB7, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, or TNFR2 costimulatory domain.

In some embodiments, the costimulatory domain is a CD28 costimulatory domain.

In some embodiments, the costimulatory domain comprises an amino acid sequence which is at least about 70% identical, at least about 80% identical, at least about 90% identical, at least about 95% or about 100% identical to the sequence set forth in SEQ ID NO:36.

In some embodiments, the the transmembrane domain comprises a CD28, CD3 epsilon, CD3 zeta, CD45, CD4, CD5, CD8, CD9, CD16, CD22, CD33, CD37, CD64, CD80, CD86, CD134, CD137, CD154, KIRDS2, OX40, CD2, CD27, LFA-1 (CDl la, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGBl, CD29, ITGB2, CD18, LFA-1, ITGB7, DNAMl (CD226), SLAMF4 (CD244, 2B4), CD84, CD96 (Tactile), CEACAM1, CRT AM, Ly9 (CD229), CD160 (BY55), PSGLl, CDIOO (SEMA4D), SLAMF6 (NTB-A, Lyl08), SLAM (SLAMFl, CD150, IPO-3), BLAME (SLAMF8), SELPLG (CD162), LTBR, PAG/Cbp, NKp44, NKp30, NKp46, NKG2D, NKG2C, or TNFR2 transmembrane domain, or the alpha, beta or zeta chain of the T-cell receptor.

In some embodiments, the transmembrane domain is a CD28 transmembrane domain. In other embodiments, the transmembrane domain comprises an amino acid sequence which is at least about 70% identical, at least about 80% identical, at least about 90% identical, at least about 95% or is about 100% identical to the sequence set forth in SEQ ID NO:37.

In some embodiments, the antigen-binding domain is connected to the transmembrane domain by a hinge region. In some embodiments, the hinge region comprises a CD8 hinge, an IgG hinge, an IgD hinge, a CD28 hinge, a KIR2DS2 hinge, or a glycine-serine linker.

In some embodiments, the hinge region comprises a CD8 alpha hinge region. In some embodiments, the hinge region comprises a sequence of amino acids which is at least 70% or at least 80% or at least 90%, at least 95% or is about 100% identical to SEQ ID NO:38.

In some embodiments, the CAR further comprises a leader sequence. In some embodiments, the leader sequence is a human leader sequence. In some embodiments, the leader sequence is an N-terminal leader sequence. In some embodiments, the leader sequence is an IgG kappa leader sequence, an IgG2 heavy chain leader sequence, an IL2 leader sequence, or an IgK VIII leader sequence. In some embodiments, the leader sequence comprises a sequence of amino acids which is at least 70% or at least 80% or at least 90%, at least 95% or is about 100% identical to SEQ ID NO:39. Other suitable leader sequences will be known by those skilled in the art. In some embodiments, the leader sequence is cleaved from the CAR during cellular processing and localization of the CAR to the cellular membrane when expressed in a cell.

In some embodiments, the CAR comprises more than one antigen-binding domain. Thus, in some embodiments, the CAR is a multi-specific CAR comprising two or more antigen-binding domains, wherein one of the antigen-binding domains comprises the binding protein of the invention. The two or more antigen-binding domains may bind to the same or different targets.

In an embodiment, the CAR comprises an amino acid sequence which is at least about 70%, at least about 80%, at least about 90%, or at least about 95% identical to the sequence set forth in SEQ ID NO:25 or SEQ ID NO:26.

In an embodiment, the CAR comprises the amino acid sequence set forth in SEQ ID NO:25 or SEQ ID NO:26.

In another aspect, the present invention provides a nucleic acid encoding a binding protein of the invention or the CAR of the invention.

In an embodiment, the nucleic acid comprises a nucleotide sequence that is codon optimized for expression in human cells.

In an embodiment, the nucleic acid comprises a nucleotide sequence which is at least about 70%, at least about 80%, at least about 90%, or at least about 95% identical to any one or more of the sequences provided in SEQ ID NOs: 29, 30, 31, 32, 33 or 34.

In an embodiment, the nucleic acid comprises any one or more of the nucleotide sequences provided in SEQ ID NOs: 29, 30, 31, 32, 33 or 34.

In an embodiment, the nucleic acid comprises
i) a nucleotide sequence which is at least about 70%, at least about 80%, at least about 90%, or at least about 95% identical to SEQ ID NO:29, and
ii) a nucleotide sequence which is at least about 70%, at least about 80%, at least about 90%, or at least about 95% identical to SEQ ID NO:30.

In an embodiment, the nucleic acid comprises a nucleotide sequence which is at least about 70%, at least about 80%, at least about 90%, or at least about 95% identical to SEQ ID NO:31

In an embodiment, the nucleic acid comprises a nucleotide sequence which is at least about 70%, at least about 80%, at least about 90%, or at least about 95% identical to SEQ ID NO:33.

In an embodiment, the nucleic acid comprises a nucleotide sequence which is at least about 70%, at least about 80%, at least about 90%, or at least about 95% identical to SEQ ID NO:34.

In another aspect, the present invention provides a vector comprising the nucleic acid of the invention. In an embodiment, the vector is a DNA vector, an RNA vector, a plasmid, a lentivirus vector, adenovirus or adeno-associated virus vector, or a retrovirus vector. In some embodiments, the nucleic acid of the invention is operably linked to a promoter in the vector.

In another aspect, the present invention provides a cell comprising the binding protein of the invention, the CAR of the invention, the nucleic acid of the invention, or the vector of the invention. In a similar aspect, the present invention provides a cell expressing the binding protein or CAR of the invention.

In some embodiments, the cell is an immune effector cell. In some embodiments, the immune effector cell is a T cell or an NK cell. In some embodiments, the immune effector cell is a CD8+ T cell. In some embodiments, the immune effector cell is a CD4+ T cell. In some embodiments, the cell is a mammalian cell. In some embodiments, the cell is a human cell.

In one embodiment, the cell described herein can further comprise a second (or more) CAR, e.g., a second CAR that includes a different antigen-binding domain, e.g., to the same target (i.e., EGFRvIII) or a different target. In one embodiment, the second CAR includes an antigen-binding domain which binds to a target expressed on the same cancer cell type as the target of the first CAR.

In one embodiment, the second CAR in the cell is an inhibitory CAR, wherein the inhibitory CAR comprises an antigen-binding domain, a transmembrane domain, and an intracellular domain of an inhibitory molecule. In one embodiment, the inhibitory CAR comprises an antigen-binding domain that binds a target found on normal cells but not disease cells, e.g., normal cells that also express the target of the first CAR. The inhibitory molecule can be chosen from one or more of: PD1, PD-L1, CTLA-4, TIM-3, LAG-3, VISTA, BTLA, TIGIT, LAIR1, CD160, 2B4, TGF beta, CEACAM-1, CEACAM-3, and CEACAM-5. In one embodiment, the second CAR comprises the extracellular domain of PD1 or a fragment thereof.

In another aspect, the present invention provides a composition comprising the nucleic acid of the invention, the vector of the invention, or the cell of the invention and a pharmaceutically acceptable carrier.

In another aspect, the present invention provides a method of making a CAR-expressing cell, comprising introducing the nucleic acid of the invention or the vector of the invention, into a cell, under conditions such that the CAR is expressed.

In some instances, it may be advantageous to reduce the amount of or remove T regulatory cells before introducing the nucleic acid or vector into the cells. Thus, in some embodiments, the method of making a CAR-expressing cell comprises:
a) providing a population of cells comprising T regulatory cells; and
b) removing T regulatory cells from the population, thereby providing a population of T regulatory-depleted cells; and
c) introducing the nucleic acid or the vector of the invention into the population of T regulatory-depleted cells such that the CAR is expressed. Advantageously, such methods can be used to produce a population of CAR-expressing cells which are immune effector cells, such as CD4+ and CD8+ T cells.

In some embodiments, the T regulatory cells are removed from the population of cells using an anti-CD25 antibody, or an anti-GITR antibody.

In certain embodiments, the method further comprises expanding the population of cells after the nucleic acid molecule encoding a CAR has been introduced.

In some embodiments, the population of cells is expanded by culturing the cells in the presence of an agent that stimulates a CD3/TCR complex associated signal and/or a ligand that stimulates a costimulatory molecule on the surface of the cells. The agent can be a bead conjugated with anti-CD3 antibody, or a fragment thereof, and/or anti-CD28 antibody, or a fragment thereof.

In some embodiments, the population of cells is expanded in an appropriate media that includes one or more interleukin that result in at least a 200-fold, 250-fold, 300-fold, or 350-fold increase in cells over a 14 day expansion period, as measured by flow cytometry.

In other embodiments, the population of cells is expanded in the presence IL-15 and/or IL-7.

In certain embodiments, the method further includes cryopreserving the population of the cells after expanding them.

In another aspect, the present invention provides a method of treating a subject having a cancer associated with expression of EGFRvIII, the method comprising administering to the subject the binding protein of the invention, the composition of the invention, or the cell of the invention.

In a related aspect, the present invention provides the binding protein of the invention, the composition of the invention, or the cell of the invention, for use in the treatment of a cancer associated with expression of EGFRvIII.

In another related aspect, the present invention provides use of the binding protein of the invention, the composition of the invention, or the cell of the invention in the manufacture of a medicament for the treatment of a cancer associated with expression of EGFRvIII.

In some embodiments, a population of cells of the invention are administered. In some embodiments, the cells administered to the subject are allogenic cells or autologous cells. In some embodiments, the cells lack or have low expression of a functional T cell receptor (TCR) or a functional human leukocyte antigen (HLA). The phrase “low expression”, in this context, refers to an amount of expression that is insufficient to affect cell signalling, e.g., an amount of expression of a TCR which is insufficient to activate the cell in the presence of its antigen. Advantageously, such cells are specific for the antigen recognized by the CAR.

In some embodiments, the treatment comprises administering an agent that increases the efficacy of the cells. In some embodiments, the agent is chosen from one or more of:
a) a protein phosphatase inhibitor;
b) a kinase inhibitor;
c) a cytokine;
d) an inhibitor of an immune inhibitory molecule; or
e) an agent that decreases the level or activity of T regulatory cells.

In some embodiments, the cancer associated with expression of EGFRvIII is lung cancer, breast cancer, colorectal cancer, prostate cancer, ovarian cancer, skin cancer, melanoma, stomach cancer, pancreatic cancer, liver cancer, brain cancer, glioblastoma, neuroblastoma, blood cancer, acute myeloid leukaemia, acute lymphoblastic leukaemia, chronic lymphocytic leukemia, chronic myelogenous leukemia, chronic myeloproliferative neoplasms, parathyroid cancer, renal cancer, retinoblastoma, rhabdomyosarcoma, savilary gland cancer, sarcoma, T cell lymphoma, throat cancer, thymoma, thymic carcinoma, Wilms tumour, Hodgkin lymphoma, non-Hodgkin lymphoma, Merkel cell carcinoma, esophageal cancer, bladder cancer, bile duct cancer, bone cancer, Ewing sarcoma, osteosarcoma, malignant fibrous histiocytoma, or multiple myeloma.

In some embodiments, the cancer associated with expression of EGFRvIII is glioma, medulloblastoma, breast cancer, ovarian cancer, colon cancer, lung cancer, or prostate cancer. In some embodiments, the cancer is glioblastoma; breast cancer, ovarian cancer, lung cacner; head and neck squamous cell carcinoma; medulloblastoma, colorectal cancer, prostate cancer, or bladder cancer.

In some embodiments, the cancer associated with expression of EGFRvIII is glioblastoma (also known as glioblastoma multiforme, GBM).

In some embodiments, the subject is a mammal. In some embodiments, the subject is a human. In other embodiments, the subject is a non-human mammal.

As described herein, the present inventors have found that, in some embodiments, the binding protein or cell of the invention are also effective against cancer cells which overexpress EGFR, e.g., due to a gene amplification. Thus, in another aspect, the present invention provides a method of treating a subject having a cancer associated with overexpression of EGFR, the method comprising administering to the subject the binding protein of the invention, the composition of the invention, or the cell of the invention.

In a related aspect, the present invention provides the binding protein of the invention, the composition of the invention, or the cell of the invention, for use in the treatment of a cancer associated with overexpression of EGFR.

In an another related aspect, the present invention provides use of the binding protein of the invention, the composition of the invention, or the cell of the invention in the manufacture of a medicament for the treatment of a cancer associated with overexpression of EGFR.

In some embodiments, the cancer associated with overexpression of EGFR is lung cancer, breast cancer, colorectal cancer, prostate cancer, ovarian cancer, skin cancer, melanoma, stomach cancer, pancreatic cancer, liver cancer, brain cancer, glioblastoma, neuroblastoma, blood cancer, acute myeloid leukaemia, acute lymphoblastic leukaemia, chronic lymphocytic leukemia, chronic myelogenous leukemia, chronic myeloproliferative neoplasms, parathyroid cancer, renal cancer, retinoblastoma, rhabdomyosarcoma, savilary gland cancer, sarcoma, T cell lymphoma, throat cancer, thymoma, thymic carcinoma, Wilms tumour, Hodgkin lymphoma, non-Hodgkin lymphoma, Merkel cell carcinoma, esophageal cancer, bladder cancer, bile duct cancer, bone cancer, Ewing sarcoma, osteosarcoma, malignant fibrous histiocytoma, or multiple myeloma.

In some embodiments, the cancer associated with overexpression of EGFR is glioma, pancreatic cancer, colorectal cancer, lung cancer, breast cancer, stomach cancer, renal cancer, cervical cancer, ovarian cancer, adenocarcinoma, bladder cancer, or head and neck cancer.

In some embodiments, the cancer associated with overexpression of EGFR is glioblastoma.

In another aspect, the present invention provides a method of detecting a cell expressing EGFRvIII, the method comprising contacting the binding protein of the invention with a sample comprising the cell and detecting binding between the binding protein and EGFRvIII.

In a related aspect, the present invention provides a method of diagnosing a subject with a cancer associated with expression of EGFRvIII, the method comprising
a) obtaining a sample comprising cells from the subject;
b) determining whether the cells express EGFRvIII by contacting the sample with the binding protein of the invention and detecting binding between the binding protein and EGFRvIII; and
c) diagnosing the subject with the cancer associated with expression of EGFRvIII if binding is detected in step b).

In another aspect, the present invention provides a method of detecting a cell overexpressing EGFR, the method comprising contacting the binding protein of the invention with a sample comprising the cell and detecting binding between the binding protein and EGFR.

In a related aspect, the present invention provides a method of diagnosing a subject with a cancer associated with overexpression of EGFR, the method comprising
a) obtaining a sample comprising cells from the subject;
b) determining whether the cells overexpress EGFR by contacting the sample with the binding protein of the invention and detecting binding between the binding protein and EGFR; and
c) diagnosing the subject with the cancer associated with overexpression of EGFR if binding is detected in step b).

In another aspect, the present invention provides a method of activating an immune effector cell comprising the CAR of the invention, the method comprising contacting the immune effector cell with EGFRvIII.

In some embodiments, the immune effector cell is contacted with EGFRvIII in a subject. In some embodiments, the method further comprises administering the immune effector cell to the subject. In some embodiments, the immune effector cell is contacted with EGFRvIII in vitro.

Any embodiment herein shall be taken to apply mutatis mutandis to any other embodiment unless specifically stated otherwise. For instance, as the skilled person would understand, examples of binding proteins outlined above equally apply to the CARs, nucleic acids, vectors, and methods of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE ACCOMPANYING DRAWINGS

FIG. 1—Screening of initial anti-EGFRvIII scFv clones. Thirteen high affinity anti-EGFRvIII scFv clones identified using a display library were assessed for their specificity for EGFRvIII relative to wild type EGFR. Biotinylated scFv was first bound to streptavidin Dynabeads, which were then washed and blocked with free biotin, before binding to soluble EGFRvIII or EGFR that was labelled with ATTO488 (fluorescence on y-axis, where increased fluorescence indicates increased binding). An unrelated biotinylated scFv was used as a negative control (bars labelled “1” and “2”). Bars 3 to 28 correspond to the thirteen different anti-EGFRvIII scFv clones, with odd numbers corresponding to binding to EGFRvIII and even numbers corresponding to WT EGFR bidning. The two scFv's that showed the highest specificity for EGFRvIII, relative WT EGFR, were GCT01 (bars 3 and 4) and GCT02 (bars 13 and 14).

FIG. 2—Analysis of surface plasmon resonance data to determine the specificity and affinity of GCT01 and GCT02. Surface plasmon resonance (SPR) data showing the response units of the GFP, GCT01 and GCT02 scFv against EGFR protein (top) and EGFRvIII protein (bottom).

FIG. 3—Analysis of binding to EGFRvIII expressed on the surface of cells. Flow cytometry showing binding of recombinant GCT01 and GCT02 to EGFRvIII or EGFR protein expressed on the surface of a panel of human cell lines.

FIG. 4—Transduction efficiencies of GCT01 and GCT02 on the primary T cell surface. Transduced primary T cells were analyzed by flow cytometry 24 hrs after transduction to determine transgene construct expression (mCherry, X-axis) and by labelling with anti-MYC to determine cell surface expression (Y-axis). Shown are representative flow cytometry plots showing GCT01 and GCT02 CAR expression.

FIG. 5—GCT01 and GCT02 kill EGFRvIII expressing tumour cells in vitro Chromium-release assay of day 8 post-transduction primary mouse CD8+ T cells expressing either GCT01 or a GCT02 CAR, in culture with Cr-labelled target cells at 10:1 effector:target ratio for 24 hrs. Cytotoxicity was measured after 24 hrs, excepted for cell lines marked with “**” which were measured after 4 hrs. Shown is mean+/−SD. Data measured in triplicate. Representative of three experiments.

FIG. 6—GCT01 CAR T cells induce rapid and complete clearance of in vivo sub cutaneous lung cancer tumours.

Tumour growth curve of sub cutaneous HCC827 lung cancer tumours treated at day 7 post implantation with IV adoptive transfer of 10c6 (1CD4:1CD8) GCT01 CAR T cells or PBS−/−. Tumour size is measured 3 times weekly using digital calipers. N=5 NSG mice per group. Data representative of four experiments.

FIG. 7—GCT01 and GCT02 CART cells induce tumour regression of intracranial glioma tumours. (A) tumour growth curves of intracranial U87WT.EGFRvIII ns GFP_Luc tumours in NSG mice, administered 1CD4:1CD8 CAR T cells, as measured by weekly bioluminescence imaging. Tumour size is measured in radiance. N=5 mice per group. Each line represents a single mouse. (B) Weekly IVIS imaging demonstrates a reduction in tumour burden in mice treated with GCT01 and GCT02 CAR T cell infusions. Weeks are indicative of post CAR T cells infusion. Data representative of two experiments.

### KEY TO THE SEQUENCE LISTING

SEQ ID NO:1—Human wild-type EGFR amino acid sequence
SEQ ID NO:2—Human EGFRvIII amino acid sequence
SEQ ID NO:3—GCT01 VH amino acid sequence
SEQ ID NO:4—GCT01 VL amino acid sequence
SEQ ID NO:5—GCT02 VH amino acid sequence
SEQ ID NO:6—GCT02 VL amino acid sequence
SEQ ID NO:7—HCDR1 consensus amino acid sequence based on GCT01 and GCT02
SEQ ID NO:8—HCDR2 consensus amino acid sequence based on GCT01 and GCT02
SEQ ID NO:9—HCDR3 consensus amino acid sequence based on GCT01 and GCT02
SEQ ID NO:10—LCDR1 consensus amino acid sequence based on GCT01 and GCT02
SEQ ID NO:11—LCDR2 consensus amino acid sequence based on GCT01 and GCT02
SEQ ID NO:12—LCDR3 consensus amino acid sequence based on GCT01 and GCT02
SEQ ID NO:13—GCT01 HCDR1 amino acid sequence
SEQ ID NO:14—GCT01 HCDR2 amino acid sequence
SEQ ID NO:15—GCT01 HCDR3 amino acid sequence
SEQ ID NO:16—GCT01 LCDR1 amino acid sequence
SEQ ID NO:17—GCT01 LCDR2 amino acid sequence
SEQ ID NO:18—GCT01 LCDR3 amino acid sequence
SEQ ID NO:19—GCT02 HCDR1 amino acid sequence
SEQ ID NO:20—GCT02 HCDR2 amino acid sequence
SEQ ID NO:21—GCT02 HCDR3 amino acid sequence
SEQ ID NO:22—GCT02 LCDR1 amino acid sequence
SEQ ID NO:23—GCT02 LCDR2 amino acid sequence
SEQ ID NO:24—GCT02 LCDR3 amino acid sequence
SEQ ID NO:25—GCT01 CAR amino acid sequence
SEQ ID NO:26—GCT02 CAR amino acid sequence
SEQ ID NO:27—GCT01 complete scFv amino acid sequence
SEQ ID NO:28—GCT02 complete scFv amino acid sequence
SEQ ID NO:29—GCT01 VH DNA sequence
SEQ ID NO:30—GCT01 VL DNA sequence
SEQ ID NO:31—GCT02 VH DNA sequence
SEQ ID NO:32—GCT02 VL DNA sequence
SEQ ID NO:33—GCT01 complete scFv DNA sequence
SEQ ID NO:34—GCT02 complete scFv DNA sequence
SEQ ID NO:35—CD3 zeta primary signalling domain amino acid sequence
SEQ ID NO:36—CD28 costimulatory domain amino acid sequence
SEQ ID NO:37—CD28 transmembrane domain amino acid sequence
SEQ ID NO:38—CD8 alpha hinge region amino acid sequence
SEQ ID NO:39—An exemplary CAR N-terminal leader amino acid sequence
SEQ ID NO:40—A non-signalling EGFRvIII protein
SEQ ID NO:41—Alternative GCT01 HCDR1 amino acid sequence
SEQ ID NO:42—Alternative GCT01 HCDR2 amino acid sequence
SEQ ID NO:43—Alternative GCT01 HCDR3 amino acid sequence
SEQ ID NO:44—Alternative GCT01 LCDR1 amino acid sequence
SEQ ID NO:45—Alternative GCT01 LCDR2 amino acid sequence
SEQ ID NO:46—Alternative GCT01 LCDR3 amino acid sequence
SEQ ID NO:47—Alternative GCT02 HCDR1 amino acid sequence
SEQ ID NO:48—Alternative GCT02 HCDR2 amino acid sequence
SEQ ID NO:49—Alternative GCT02 HCDR3 amino acid sequence
SEQ ID NO:50—Alternative GCT02 LCDR1 amino acid sequence
SEQ ID NO:51—Alternative GCT02 LCDR2 amino acid sequence
SEQ ID NO:52—Alternative GCT02 LCDR3 amino acid sequence

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in immunology, molecular biology, CAR-T design and therapy, cancer therapy, pharmacology, protein chemistry, and biochemistry).

Unless otherwise indicated, the techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical linker or a disulphide bond, for example. Examples of non-covalent bonds include hydrogen bonds, ionic bonds, Van der Waals forces, and hydrophobic interactions. In some embodiments the protein is an isolated protein.

In other embodiments, the protein may be a binding protein. Typically, a “binding protein” as used herein is to be understood to be a protein that comprises an antigen binding site, useful for example, for binding to EGFRvIII.

The term “isolated protein” or “isolated binding protein” is a protein that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art.

As used herein, the term “binds” in reference to the interaction of a binding protein or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the binding protein, will reduce the amount of labeled “A” bound to the antibody.

As used herein, the term “specifically binds” or “binds specifically” shall be taken to mean that a binding protein of the disclosure reacts or associates more frequently, more rapidly, with greater duration and/or with greater affinity with a particular antigen or cell expressing same than it does with alternative antigens or cells. For example, a binding protein binds to EGFRvIII with materially greater affinity (e.g., 20 fold or 40 fold or 60 fold or 80 fold to 100 fold or 150 fold or 200 fold) than it does to other complement components or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “does not detectably bind” shall be understood to mean that a binding protein, e.g., an antibody, binds to a candidate antigen at a level less than 10%, or 8% or 6% or 5% above background. The background can be the level of binding signal detected in the absence of the binding protein and/or in the presence of a negative control binding protein (e.g., an isotype control antibody) and/or the level of binding detected in the presence of a negative control antigen. The level of binding is detected using biosensor analysis (e.g. Biacore) in which the binding protein is immobilized and contacted with an antigen, or vice versa.

For the purposes of clarification and as will be apparent to the skilled artisan based on the exemplified subject matter herein, reference to “affinity” herein is a reference to a level of binding which can be quantified using, for example, a dissociation constant (K_{D}). Generally, reference to a level of affinity of a binding protein described herein is a reference to the protein's K_{D} for a particular antigen. Thus, as referred to herein, a binding protein that has an affinity for EGFRvIII of at least 15 nM, has a K_{D} of at least 15 nM (15 nM or stronger), i.e., the numerical value of the dissociation constant is either 15 nM or lower (for example, 10 nM or 100 pM). In this regard, reference to a higher affinity is reference to K_{D} having a lower numerical value and vice versa.

As used herein, the term “epitope” (syn. “antigenic determinant”) shall be understood to mean a region of EGFRvIII to which a binding protein of the disclosure binds. This term is not necessarily limited to the specific residues or structure to which the binding protein makes contact. For example, this term includes the region spanning amino acids contacted by the binding protein and/or 5-10 or 2-5 or 1-3 amino acids outside of this region. In some embodiments, the epitope comprises a series of discontinuous amino acids that are positioned close to one another when EGFRvIII is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that a binding protein of the disclosure (or an antigen binding site thereof) reduces or prevents binding of a recited antibody or binding protein to EGFRvIII. This may be due to the binding protein (or antigen binding site) and antibody binding to the same or an overlapping epitope. It will be apparent from the foregoing that the binding protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the binding protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to EGFRvIII either in the presence or absence of the binding protein. If less antibody binds in the presence of the binding protein than in the absence of the binding protein, the binding protein is considered to competitively inhibit binding of the antibody. In one embodiment, the competitive inhibition is not due to steric hindrance.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signalling domain that is derived from a CD3 zeta molecule, the intracellular signalling domain retains sufficient CD3 zeta structure such that is has the required function, namely, the ability to generate a signal under the appropriate conditions. It does not connotate or include a limitation to a particular process of producing the intracellular signalling domain, e.g., it does not mean that, to provide the intracellular signalling domain, one must start with a CD3 zeta sequence and delete unwanted sequence, or impose mutations, to arrive at the intracellular signalling domain. The compositions and methods of the present invention encompass polypeptides and nucleic acids having the sequences specified, or sequences substantially identical or similar thereto, e.g., sequences at least 85%, 90%, or 95% identical or higher to the sequence specified.

As used herein, the term “subject” can be any animal. In one embodiment, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one embodiment, the mammal is a human.

The terms “treating” or “treatment” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a drug) or (ii) instruct a third party to treat a subject according to a claimed method. Also encompassed within the meaning of the term “treating” or “treatment” are prevention or reduction of the disease to be treated, e.g., by administering a therapeutic at a sufficiently early phase of disease to prevent or slow its progression.

The terms “co-administration” or “administered in combination” or the like, as used herein, are meant to encompass administration of the selected therapeutic agents to a single subject, and are intended to include treatment regimens in which the agents are administered by the same or different route of administration or at the same or different time.

### EGFR and EGFRvIII

The epidermal growth factor receptor (EGFR; ErbB-1; HER1 in humans) is a transmembrane protein that is a receptor for members of the epidermal growth factor family (EGF family) of extracellular protein ligands. The amino acid sequence of wild type human EGFR is provided in SEQ ID NO:1.

EGFR is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). Mutations, amplifications, or misregulations affecting expression or activity of EGFR result in many different types of cancer.

The term “EGFRvIII”, (also known as de2-7EGFR and ΔEGFR), as used herein, refers to a mutant form of EFGFR which is referred to as an EGFR class III variant, or a biologically active fragment thereof, which exhibits any characteristics specific for EGFRvIII, as opposed to those in common with normally expressed EGFR. The amino acid sequence of human EGFRvIII is provided in SEQ ID NO:2. EGFRvIII lacks amino acid residues 6 through 273 of mature EGFR and contains a new glycine residue at position 6 between amino acid residues 5 and 274.

Aberrant EGFR (as opposed to EGFRvIII) expression, often due to gene amplification, is also associated with cancer and has been observed in many epithelial tumours. Without wishing to be bound by theory, it is understood that overexpressed EGFR, such as that produced by cancer cells, has an epitope exposed which is not present in EGFR expressed by wild-type cells. Thus, a unique conformational form of the EGFR protein can be found on tumorigenic, hyperproliferative or abnormal cells but is not detectable or transitional in normal or wild-type cells, even though they are encoded by the same gene. Commercially available cell lines which overpress EGFR include U87, A431 and U251 cells. Advantageously, in some embodiments, the binding protein of the invention binds to EGFRvIII as well as EGFR which is present on cancer cells but which is not present on wild-type cells.

### Antibodies

In one embodiment, a binding protein as described herein is an antibody or a fragment thereof.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc) region, in the case of a heavy chain. A V_{H} and a V_{L} interact to form a Fv comprising an antigen binding site that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk (Chothia et al., 1987) and/or Al-Lazikani (Al-Lazikani et al., 1997); the IMGT numbering system described in Lefranc et al. (2003); or the AHO numbering system described in Honnegher and Plükthun (2001). For example, according to the numbering system of Kabat, V_{H} framework regions (FRs) and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one embodiment, reference herein to a CDR (or a FR) is in respect of those regions according to the Kabat numbering system.

“Framework regions” (FRs) are those variable region residues other than the CDR residues.

As used herein, the term “Fv” shall be taken to mean any binding protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any binding protein of the disclosure) may have multiple antigen binding sites which may or may not bind the same antigen. This term shall be understood to encompass fragments directly derived from an antibody as well as binding proteins corresponding to such a fragment produced using recombinant means. In some embodiments, the V_{H} is not linked to a heavy chain constant domain (C_{H}) 1 and/or the V_{L} is not linked to a light chain constant domain (C_{L}). Exemplary Fv containing polypeptides or binding proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “antigen binding domain” shall be taken to mean a region of an antibody that is capable of specifically binding to an antigen, that is, a VH or a VL or an Fv comprising both a VH and a VL. The antigen binding domain need not be in the context of an entire antibody, for example, it can be in isolation (e.g., a domain antibody) or in another form (e.g., scFv). Typically, an antigen binding domain, as used herein, may comprise an antigen binding site which is capable of binding or specifically binding to an antigen.

As used herein, the term “antigen binding site” shall be taken to mean a structure formed by a binding protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a V_{L} and a V_{H} that interact with the antigen and that are generally, however not always in the one or more of the CDRs in each variable region. In some embodiments, the antigen binding site comprises one or more CDRs. In some embodiments, the antigen binding site comprises three CDRs. In some embodiments, the antigen binding site comprises six CDRs. In some embodiments, an antigen binding site comprises a V_{H} or a V_{L} or a Fv.

Methods for generating antibodies are known in the art and/or described in Harlow and Lane (editors) Antibodies: A Laboratory Manual, Cold Spring Harbor Laboratory, (1988). Generally, in such methods EGFRvIII or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mabs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265.

For example, a suitable animal is immunized with an immunogen under conditions sufficient to stimulate antibody producing cells. Rodents such as rabbits, mice and rats are exemplary animals. Mice genetically engineered to express human antibodies and, for example, do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like). Alternatively, ABL-MYC technology (NeoClone, Madison WI 53713, USA) is used to produce cell lines secreting MAbs.

Antibodies can also be produced or isolated by screening a display library, e.g., a phage display library, e.g., as described in U.S. Pat. Nos. 6,300,064 and/or 5,885,793.

In another example, a phage display library is screened or an animal is immunized with EGFRvIII, or a fragment thereof, and identified binding proteins and/or antibodies are screened to identify those that are cross-reactive with EGFRvIII and/or the fragment thereof.

In a further example, EGFRvIII, or a fragment thereof, is contacted with GCT01 or GCT02 (as described in Example 2). A phage display library is then brought into contact with EGFRvIII or the fragment thereof and phage expressing proteins that can compete with GCT01 or GCT02 for binding are selected.

In a still further example, a chimeric protein comprising, e.g., a mouse EGFRvIII in which an epitope of interest from human EGFRvIII is substituted for the corresponding mouse sequence. This chimeric protein is then used to immunize mice (which are less likely to induce an immune response against the mouse protein) and/or to screen a phage display library. The resulting antibodies/proteins are then screened to identify those that bind to human EGFRvIII (particularly at the epitope of interest) and not mouse EGFRvIII.

The antibody of the present invention may be a synthetic antibody. For example, the antibody is a chimeric antibody, a humanized antibody, a human antibody or a de-immunized antibody.

### Chimeric Proteins

In one embodiment, a binding protein described herein is a chimeric protein (e.g., a chimeric antibody). The term “chimeric protein” refers to binding proteins in which a portion of the protein (e.g., heavy and/or light chain of an antibody) is identical with or homologous to corresponding sequences in proteins derived from a particular species (e.g., murine, such as mouse) or belonging to a particular protein class or subclass, while the remainder of the protein is identical with or homologous to corresponding sequences in proteins derived from another species (e.g., primate, such as human) or belonging to another protein class or subclass. For example, chimeric antibodies can utilize non-human, e.g. rodent or rabbit, variable regions and human constant regions, in order to produce an antibody with predominantly human domains. Methods for producing chimeric antibodies are described in, e.g., U.S. Pat. Nos. 4,816,567; and 5,807,715.

### Humanized and Human Proteins

The binding proteins or antibodies of the present invention may be humanized or human.

The term “humanized protein” or “humanized binding protein” shall be understood to refer to a subclass of chimeric proteins having an antigen binding site or variable region derived from a protein from a non-human species and the remaining protein structure based upon the structure and/or sequence of a human protein. For example, in a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human proteins or parts thereof (e.g., variable regions) are known in the art. Humanization can be performed following the method of U.S. Pat. Nos. 5,225,539, or 5,585,089. Other methods for humanizing a protein are not excluded.

The term “human protein” or “human binding protein” as used herein refers to proteins which have an amino acid sequence which corresponds to sequences found in humans, e.g. in the human germline or somatic cells. The “human” proteins can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4, 5 or 6 of the residues of the protein, e.g. in 1, 2, 3, 4, 5 or 6 of the residues making up one or more of the CDRs of an antibody). These “human proteins” do not actually need to be produced by a human, rather, they can be produced using recombinant means and/or isolated from a transgenic animal (e.g., mouse) comprising nucleic acid encoding human proteins. Human antibodies can be produced using various techniques known in the art, including phage display libraries (e.g., as described in U.S. Pat. No. 5,885,793).

Human antibodies which recognize a selected epitope can also be generated using a technique referred to as “guided selection.” In this approach a selected non-human monoclonal antibody, e.g., a mouse antibody, is used to guide the selection of a completely human antibody recognizing the same epitope (e.g., as described in U.S. Pat. No. 5,565,332).

Exemplary human proteins are described herein and include GCT01 and GCT02. These human proteins provide an advantage of reduced immunogenicity in a human compared to non-human proteins.

### Other Antigen Binding Site Containing Binding Proteins

### Single-Domain Antibodies

In some embodiments, a binding protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (see, e.g., U.S. Pat. No. 6,248,516).

### Diabodies, Triabodies, Tetrabodies

In some embodiments, a binding protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR. in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### BiTEs (Bispecific T Cell Engagers)

As is known in the art, a BiTE generally refers to a single polypeptide chain molecule that has two antigen binding sites, one of which binds to an immune effector cell antigen (e.g., CD3) and the second of which binds to an antigen present on the surface of a target cell, e.g., EGFRvIII. BiTEs and methods of their production are described in WO 05/061547, Baeuerle et al. (2008), and Bargou et al. (2008).

When both targets are engaged, the BiTE molecule forms a bridge between the cytotoxic T cell and the tumor cell, which enables the T cell to recognize the tumor cell and fight it through an infusion of toxic molecules. The tumor-binding arm of the molecule can be altered to create different BiTE antibody constructs that target different types of cancer. BiTEs are typically produced as recombinant, glycosylated proteins secreted by higher eukaryotic cell lines. Accordingly, in another embodiment of this invention, the protein of the invention is a BiTE.

### Other Antigen Binding Proteins

The present invention also includes other binding proteins comprising antigen binding sites such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### De-Immunized Antibodies and Proteins

In some embodiments, the binding protein of the invention is a de-immunized antibody or protein. De-immunized antibodies and proteins have one or more epitopes, e.g., B cell epitopes or T cell epitopes removed (i.e., mutated) to thereby reduce the likelihood that a mammal will raise an immune response against the antibody or protein. Methods for producing de-immunized antibodies and proteins are known in the art and described, for example, in WO00/34317, WO2004/108158 and WO2004/064724.

Methods for introducing suitable mutations and expressing and assaying the resulting protein will be apparent to the skilled artisan based on the description herein.

### Constant Regions

The present invention also encompasses binding proteins described herein comprising a constant region of an antibody. This includes antigen binding fragments of an antibody fused to a Fc as well as full length antibodies.

Sequences of constant regions useful for producing the binding proteins of the present invention may be obtained from a number of different sources. In some embodiments, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one embodiment, the constant region is human isotype IgG4 or a stabilized IgG4 constant region.

In one embodiment, the Fc region of the constant region has a reduced ability to induce effector function, e.g., compared to a native or wild-type human IgG1 or IgG3 Fc region. In one embodiment, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one embodiment, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one embodiment, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody.

### Mutations to Proteins

The present invention also encompasses mutant forms of binding proteins described herein. For example, substitions may be made within a CDR of a binding protein of the invention and within a FR of a variable region containing protein without inhibiting or significantly reducing its function in the context of the present invention. In this regard, amino acid substitutions may be introduced into a binding protein of the invention and the products screened for a desired activity, e.g., retained/improved antigen binding, decreased immunogenicity, or improved ADCC or CDC.

For example, such a mutant binding protein comprises one or more conservative amino acid substitutions compared to a sequence set forth herein. In some embodiments, the binding protein comprises 30 or fewer or 20 or fewer or 10 or fewer, e.g., 9 or 8 or 7 or 6 or 5 or 4 or 3 or 2 conservative amino acid substitutions. A “conservative amino acid substitution” is one in which the amino acid residue is replaced with an amino acid residue having a similar side chain and/or hydropathicity and/or hydrophilicity.

In one embodiment, a mutant binding protein has only, or not more than, one or two or three or four or five or six conservative amino acid changes when compared to a naturally occurring protein. Details of conservative amino acid changes are provided below. As the skilled person would be aware, e.g., from the disclosure herein, such minor changes can reasonably be predicted not to alter the activity of the binding protein.

In some embodiments, the binding protein has no more than 3, no more than 2, or no more than 1 amino acid substitution in the CDR-L1, no more than 2 or no more than 1 amino acid substitutions in the CDR-L2, no more than 3, no more than 2, or no more than 1 amino acid substitution in the CDR-L3, no more than 2 or no more than 1 amino acid substitution in the CDR-H1, no more than 4, no more than 3, no more than 2, or no more than 1 amino acid substitution in the CDR-H2, and/or no more than 3, no more than 2, or no more than 1 amino acid substitution in the CDR-H3, relative to any one or more of the CDR amino acid sequences provided herein. In some embodiments, each CDR contains no more than one, two, three, or four amino acid substitutions. In some embodiments, the amino acid substitutions are conservative substitutions. In some embodiments, the binding protein comprises an amino acid substitution in a framework region. As a person skilled in the art would appreciate, routine site-directed or random mutagenesis techniques can be performed to alter the amino acid sequence of any one of the CDR or framework sequences provided herein in order to, for example, alter binding affinity (e.g., affinity maturation), reduce susceptibility to proteolysis or oxidation, or confer or modify other physicochemical or functional properties of the binding proteins.

Examples of conservative amino acid substitutions are provided below in Table 1.

TABLE 1
Exemplary conservative amino acid changes

| Original | | Preferred |
|---|---|---|
| amino acid | Exemplary substitutions | substitutions |
| Ala | Val; Leu; Ile | Val |
| Arg | Lys; Gln; Asn | Lys |
| Asn | Gln; His; Asp, Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Ala | Ala |
| His | Asn; Gln; Lys; Arg | Arg |
| Ile | Leu; Val; Met; Ala; Phe; Norleucine | Leu |
| Leu | Norleucine; Ile; Val; Met; Ala; Phe | Ile |
| Lys | Arg; Gln; Asn | Arg |
| Met | Leu; Phe; Ile | Leu |
| Phe | Trp; Leu; Val; Ile; Ala; Tyr | Tyr |
| Pro | Ala | Ala |
| Ser | Thr | Thr |
| Thr | Val; Ser | Ser |
| Trp | Tyr; Phe | Tyr |
| Tyr | Trp; Phe; Thr; Ser | Phe |
| Val | Ile; Leu; Met; Phe; Ala; Norleucine | Leu |

The present invention also contemplates non-conservative amino acid changes (e.g., substitutions) in a binding protein of the present invention, e.g., in a CDR, such as CDR3. In one embodiment, the binding protein comprises fewer than 6 or 5 or 4 or 3 or 2 or 1 non-conservative amino acid substitutions, e.g., in a CDR3, such as in a CDR3.

Routine techniques can be used to introduce amino acid substitutions in CDRs to, for example, improve binding affinity. Such substitutions may be made in CDR “hotspots,” i.e., residues encoded by codons that undergo mutation at high frequency during the somatic maturation process (see, e.g., Chowdhury, 2008)), and/or residues that contact EGFRvIII, with the resulting variant V_{H} and/or V_{L} being tested for binding affinity. Affinity maturation, error-prone PCR, chain shuffling, or oligonucleotide-directed mutagenesis are commonly used techniques.

In certain examples, substitutions, insertions, or deletions may occur within one or more CDRs so long as such alterations do not substantially reduce the ability of the binding protein to bind to EGFRvIII. In some embodiments, the binding protein comprising the amino acid substitutions binds to EGFRvIII with a similar affinity to the binding protein without the substitutions. Such substitutions may, for example, be outside of antigen contacting residues in the CDRs. In some embodiments, the binding protein comprising the amino acid substitutions binds to EGFRvIII with a higher affinity than the binding protein without the substitutions. In some embodiments, the binding protein comprising the amino acid substitutions binds to EGFRvIII with a lower affinity than the binding protein without the substitutions. In certain examples, each CDR either is unaltered, or contains no more than one, two, three, or four amino acid substitutions. In some embodiments, the substitutions are conservative substitutions.

A useful method for identification of residues or regions of an antibody that may be targeted for mutagenesis is called “alanine scanning mutagenesis” as described by Cunningham (1989). In this method, a residue or group of target residues (e.g., charged residues such as Arg, Asp, His, Lys, and Glu) are identified and replaced by a neutral amino acid such as alanine to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues may be targeted or eliminated as candidates for substitution. Variants may be screened to determine whether they contain the desired properties.

The present invention also contemplates one or more insertions or deletions compared to a sequence set forth herein. In some embodiments, the binding protein comprises 10 or fewer, e.g., 9 or 8 or 7 or 6 or 5 or 4 or 3 or 2 insertions and/or deletions.

### Conjugates

In some embodiments, the binding protein of the invention is conjugated to another compound. Conjugates could help extend half-life or impart other biological activites. Methods for conjugation of the binding protein will be apparent to the skilled person and/or described herein. All forms and methods of conjugation (i.e., binding) are contemplated by the present invention, including, for example, direct conjugation between the binding protein and another compound/moiety as described herein or indirect binding (e.g., by virtue of a linker between the binding protein and the other compound/moiety). In one embodiment, the conjugate is formed by a chemical conjugation (e.g., by an amine bond or disulphide bond) or by genetic fusion.

In one embodiment, the disclosure provides a fusion protein comprising the binding protein of the invention and the other compound. For example, the other compound can be positioned at the N-terminus of the protein, C-terminus of the protein or any combination thereof.

In one embodiment, the binding protein is conjugated to the other compound via a linker. For example, the linker can be a peptide linker.

In one embodiment, the linker is a flexible linker. A “flexible” linker is an amino acid sequence which does not have a fixed structure (secondary or tertiary structure) in solution. Such a flexible linker is therefore free to adopt a variety of conformations. Flexible linkers suitable for use in the present invention are known in the art. An example of a flexible linker for use in the present invention is the linker sequence SGGGGS/GGGGS/GGGGS or (Gly4Ser)3. Flexible linkers are also disclosed in WO1999045132.

The linker may comprise any amino acid sequence that does not substantially hinder interaction of the binding region with its target. Preferred amino acid residues for flexible linker sequences include, but are not limited to, glycine, alanine, serine, threonine proline, lysine, arginine, glutamine and glutamic acid.

The linker sequences between the binding regions preferably comprise five or more amino acid residues. The flexible linker sequences according to the present invention consist of 5 or more residues, preferably, 5, 6, 7, 8, 9, 10, 11, 12, 13, 14, 15, 16, 17, 18, 19 or 20 or 25 or 30 or more residues. In an embodiment of the invention, the flexible linker sequences consist of 5, 7, 10, 13 or 16 or 30 residues.

Exemplary compounds that can be conjugated to a binding protein of the invention could be selected from the group consisting of a human serum albumin or functional fragment thereof, an immunoglobulin Fc region or functional fragment thereof, afamin, alpha-fetoprotein, vitamin D binding protein, antibody fragments that bind to albumin and polymers. Other exemplary compounds include

### Cytotoxic Agents

In an embodiment, the binding protein is conjugated to a cytotoxic agent. Cytotoxic agents include any agent that is detrimental to the growth, viability or propagation of cells. Examples of suitable cytotoxic agents and chemotherapeutic agents that can be conjugated to binding proteins in accordance with this aspect of the invention include, e.g., 1-(2chloroethyl)-1,2-dimethanesulfonyl hydrazide, 1,8-dihydroxy-bicyclo[7.3.1 ]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins, camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin, doxorubicin, duocarmycin, echinomycins, eleutherobins, emetine, epothilones, esperamicin, estramustines, ethidium bromide. etoposide, fluorouracils, geldanamycins, gramicidin D, glucocorticoids, irinotecans, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PDBs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing. According to certain embodiments, the cytotoxic agent that is conjugated to the binding protein is a maytansinoid such as DM1 or DM4, a tomaymycin derivative, or a dolastatin derivative. Other cytotoxic agents known in the art are contemplated within the scope of the present invention, including, e.g., protein toxins such ricin, C. difficile toxin, pseudomonas exotoxin, ricin, diphtheria toxin, botulinum toxin, bryodin, saporin, pokeweed toxins (i.e., phytolaccatoxin and phytolaccigenin), and others.

### Binding Protein Production

In one embodiment, a binding protein described herein according to any example is produced using methods that are known in the art, e.g., by culturing a hybridoma under conditions sufficient to produce the binding protein.

### Recombinant Expression

In another example, a binding protein described herein according to any example is recombinant.

In the case of a recombinant binding protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the binding protein. Exemplary cells used for expressing a binding protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. Nos. 4,816,567 or 5,530,101.

Following isolation, the nucleic acid is inserted operably linked to a promoter in an expression construct or expression vector for further cloning (amplification of the DNA) or for expression in a cell-free system or in cells.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

### Isolation of Proteins

Methods for isolating a binding protein are known in the art and/or described herein. For example, where a binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the binding protein, e.g., using continuous centrifugation.

The binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art. The skilled artisan will also be aware that a binding protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting binding protein is then purified using methods known in the art, such as, affinity purification.

### Assaying Activity of a Binding Protein

### Binding to EGFR and Mutants Thereof

Methods for assessing binding to a binding protein are known in the art, e.g., as described in Scopes (In: Protein purification: principles and practice, Third Edition, Springer Verlag, 1994). Such a method generally involves labeling the binding protein and contacting it with immobilized antigen or vice versa. Following washing to remove non-specific bound protein, the amount of label and, as a consequence, bound protein is detected. Of course, the binding protein can be immobilized and the antigen labeled. Panning-type assays can also be used. Alternatively, or additionally, surface plasmon resonance assays can be used. Thus, in one embodiment, the affinity of a binding protein described herein is determined using a biosensor.

Optionally, the dissociation constant (Kd) of a binding protein for EGFRvIII is determined. The “Kd” or “K_{D}” or “Kd value” for a EGFRvIII binding protein is in one embodiment measured by a radiolabeled or fluorescently-labeled EGFRvIII binding assay. This assay equilibrates the binding protein with a minimal concentration of labeled EGFRvIII in the presence of a titration series of unlabeled EGFRvIII. Following washing to remove unbound EGFRvIII, the amount of label is determined, which is indicative of the Kd of the binding protein.

According to another example the Kd is measured by using surface plasmon resonance assays, e.g., using BIAcore surface plasmon resonance (BIAcore, Inc., Piscataway, NJ) with immobilized protein of the invention or vice versa. Thus, in one embodiment, the affinity of a binding protein is determined using a biosensor (e.g., by surface plasmon resonance) in an assay in which the binding protein is immobilized and EGFRvIII is contacted with the immobilized binding protein.

### Epitope Mapping

In another example, the epitope bound by a binding protein described herein is mapped. Epitope mapping methods will be apparent to the skilled artisan. For example, a series of overlapping peptides spanning the EGFRvIII sequence or a region thereof comprising an epitope of interest, e.g., peptides comprising 10-15 amino acids are produced. The binding protein is then contacted to each peptide and the peptide(s) to which it binds determined. This permits determination of peptide(s) comprising the epitope to which the binding protein binds. If multiple non-contiguous peptides are bound by the binding protein, the binding protein may bind a conformational epitope.

Alternatively, or in addition, as exemplified herein, amino acid residues within EGFRvIII are mutated, e.g., by alanine scanning mutagenesis, and mutations that reduce or prevent protein binding are determined. Any mutation that reduces or prevents binding of the binding protein is likely to be within the epitope bound by the binding protein.

A further method involves binding EGFRvIII or a region thereof to an immobilized binding protein of the present invention and digesting the resulting complex with protcases. Peptide that remains bound to the immobilized protein are then isolated and analyzed, e.g., using mass spectrometry, to determine their sequence.

A further method involves converting hydrogens in EGFRvIII or a region thereof to deutrons and binding the resulting protein to an immobilized binding protein of the present invention. The deutrons are then converted back to hydrogen, the EGFRvIII or region thereof isolated, digested with enzymes and analyzed, e.g., using mass spectrometry to identify those regions comprising deutrons, which would have been protected from conversion to hydrogen by the binding of a binding protein described herein.

### Determining Competitive Binding

Assays for determining whether a protein competitively inhibits binding of a binding protein described herein, such as GCT01 or GCT02, will be apparent to the skilled artisan. For example, the binding protein can be conjugated to a detectable label, e.g., a fluorescent label or a radioactive label. The labeled binding protein and the test protein are then mixed and contacted with EGFRvIII or a region thereof or a cell expressing same. The level of labeled binding protein is then determined and compared to the level determined when the labeled binding protein is contacted with EGFRvIII, region or cells in the absence of the test protein. If the level of labeled binding protein is reduced in the presence of the test protein compared to the absence of the test protein, the test protein is considered to competitively inhibit binding of the labeled binding protein.

Optionally, the test protein is conjugated to a different label to the labeled binding protein. This alternate labeling permits detection of the level of binding of the test protein to EGFRvIII or the region thereof or the cell.

In another example, the binding protein is permitted to bind to EGFRvIII or a region thereof or a cell expressing same prior to contacting the EGFRvIII, region or cell with the test protein. A reduction in the amount of bound binding protein in the presence of the test protein compared to in the absence of the test protein indicates that the test protein competitively inhibits binding to EGFRvIII. A reciprocal assay can also be performed using labeled binding protein and first allowing unlabeled protein to bind to EGFRvIII. In this case, a reduced amount of labeled binding protein bound to EGFRvIII in the presence of the unlabeled protein compared to in the absence of the unlabeled protein indicates that the labeled binding protein competitively inhibits binding of the unlabeled protein to EGFRvIII.

### Determining Sequence Identity

Percent identity in the context of two or more polypeptide or nucleotide sequences, refers to the percentage of amino acids or nucleotides that are the same, within a given region of the polypeptide or nucleotide sequences. For example, two sequences may have e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a region in the sequences or, when specified, over the entire sequence, when compared and aligned for maximum correspondence over a comparison window, or designated region as measured using one of the following sequence comparison algorithms or by manual alignment and visual inspection. Optionally, the identity exists over a region that is at least about 20 amino acids in length, a region that is 30, 40, 50, 75, 100, 125, 150, 175, 200 or more amino acids in length.

Methods of alignment of sequences for comparison are well known in the art. Optimal alignment of sequences for comparison can be conducted, e.g., by the local homology algorithm of Smith and Waterman (1970), by the homology alignment algorithm of Needleman and Wunsch (1970), by the search for similarity method of Pearson and Lipman, (1988), by computerized implementations of these algorithms (e.g., GAP, BESTFIT, FASTA, and TFASTA in the Wisconsin Genetics Software Package, Genetics Computer Group), or by manual alignment and visual inspection (see, e.g., Brent et al., 2003).

Two examples of algorithms that are suitable for determining percent sequence identity and sequence similarity are the BLAST and BLAST 2.0 algorithms, which are described in Altschul et al. (1977); and Altschul et al. (1990), respectively.

The percent identity between two amino acid or nucleotide sequences can also be determined using the algorithm of Meyers and Miller (1988), which has been incorporated into the ALIGN program (version 2.0), using a PAM120 weight residue table, a gap length penalty of 12 and a gap penalty of 4. In addition, the percent identity between two amino acid or nucleotide sequences can be determined using the Needleman and Wunsch (1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2. 3, 4, 5, or 6.

### Compositions

In some embodiments, a binding protein as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing a protein into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this disclosure are particularly useful for parenteral administration, such as intravenous administration or administration into a body cavity or lumen of an organ or joint. The compositions for administration will commonly comprise a solution of protein dissolved in a pharmaceutically acceptable carrier. for example an aqueous carrier. A variety of aqueous carriers can be used. e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of proteins of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, proteins of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver compounds of the present invention.

WO2002/080967 describes compositions and methods for administering aerosolized compositions comprising antibodies for the treatment of, e.g., asthma, which are also suitable for administration of a protein of the present invention.

### Chimeric Antigen Receptors (CARs)

The term “chimeric antigen receptor” or alternatively “CAR” refers to a polypeptide or set of polypeptides, which when in an immune effector cell, provides the cell with specificity for a target cell, for example a cancer cell, and with intracellular signal generation. The CAR described herein comprises at least an extracellular antigen-binding domain comprising the binding protein of the invention, a transmembrane domain and an intracellular domain (also referred to herein as a “cytoplasmic signalling domain” or “an intracellular signalling domain”).

In some embodiments, the CAR domains are in the same polypeptide chain (e.g., comprise a chimeric fusion protein). In some embodiments, the domains are contiguous with each other. In some embodiments, the domains are not contiguous with each other, e.g., are in different polypeptide chains, such as a split CAR. In some embodiments, the different polypeptide chains include a dimerization switch that, upon the presence of a dimerization molecule, can couple the polypeptides to one another, e.g., can couple an antigen-binding domain to an intracellular signalling domain.

In some embodiments, the CAR further comprises a leader sequence. As used herein the term “leader sequence” refers to a sequence of amino acids which, when fused to a CAR sequence, aids localization of the CAR to a cell membrane of a cell expressing the CAR. In some embodiments, the leader sequence is an N-terminal leader sequence. In some embodiments, the leader sequence comprises a sequence of amino acids which is at least 70% or at least 80% or at least 90% or at least 95% identical to SEQ ID NO:39. In some embodiments, the leader sequence is cleaved from the CAR during cellular processing and localization of the CAR to the cellular membrane when expressed in a cell.

### Antigen-Binding Domains

As described herein, the CAR of the invention comprises a target-specific binding element otherwise referred to as an “antigen-binding domain”. The antigen-binding domain can be any domain that binds to EGFRvIII including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen-binding domain, such as a recombinant fibronectin domain, a T cell receptor (TCR), or a fragment thereof, e.g., single chain TCR, and the like. In some instances, it is beneficial for the antigen-binding domain to be derived from the same species in which the CAR will ultimately be used in. For example, for use in humans, it may be beneficial for the antigen-binding domain of the CAR to comprise human or humanized residues for the antigen-binding domain of an antibody or antibody fragment.

In some embodiments, the antigen-binding domain comprises a single domain antigen binding (SDAB) molecule, which include molecules whose complementary determining regions are part of a single domain polypeptide. Examples include, but are not limited to, heavy chain variable domains, binding molecules naturally devoid of light chains, single domains derived from conventional 4-chain antibodies. engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks. In one embodiment, an SDAB molecule can be derived from a variable region of the immunoglobulin found in fish, such as, for example, that which is derived from the immunoglobulin isotype known as a Novel Antigen Receptor (NAR) found in the serum of shark. Methods of producing single domain molecules derived from a variable region of NAR (“IgNARs”) are described in WO 03/014161 and Streltsov (2005).

In one embodiment, the SDAB molecule is a naturally occurring single domain antigen binding molecule known as heavy chain devoid of light chains. Such single domain molecules are disclosed in WO 9404678 and Hamers-Casterman et al. (1993) for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention. The SDAB molecules can be recombinant, CDR-grafted, humanized, camelized, de-immunized and/or in vitro generated (e.g., selected by phage display).

In one embodiment, the antigen-binding domain is characterized by particular functional features or properties of an antibody or antibody fragment. For example, in one embodiment, the portion of a CAR composition of the invention that comprises an antigen-binding domain specifically binds a tumour antigen as described herein.

In one embodiment, the antigen-binding domain is an antibody fragment, e.g., a single chain variable fragment (scFv). In one embodiment, the antigen-binding domain is a Fv, a Fab, a (Fab′)2, or a bi-functional (e.g. bi-specific) hybrid antibody (e.g., Lanzavecchia et al., 1987). In one embodiment, the antibodies and fragments thereof of the invention binds to its target with wild-type or enhanced affinity. In some instances, scFvs can be prepared according to method known in the art (see, for example, Bird et al., 1988 and Huston et al., 1988). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see Hollinger et al. 1993, US 2005/0100543, US 2005/0175606, US 2007/0014794, WO2006/020258 and WO2007/024715.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly4Ser)n, where n is a positive integer equal to or greater than 1. In one embodiment, the linker can be (Gly4Ser)4 or (Gly4Ser)3. Variation in the linker length may retain or enhance activity, giving rise to superior efficacy in activity studies.

In another embodiment, the antigen-binding domain is a T cell receptor (“TCR”), or a fragment thereof, for example, a single chain TCR (scTCR). Methods to make such TCRs are known in the art (see, e.g., Willemsen et al., 2000; Zhang et al., 2004; Aggen et al., 2012). For example, an scTCR can be engineered that contains the Va and νβ genes from a T cell clone linked by a linker (e.g., a flexible peptide). This approach is very useful to cancer associated target that itself is intracellular, however, a fragment of such antigen (peptide) is presented on the surface of the cancer cells by MHC.

Other types of suitable antigen-binding domains are discussed herein in relation to binding proteins of the invention.

### Transmembrane Domain

With respect to the transmembrane domain, in various embodiments, a CAR can be designed to comprise a transmembrane domain that is attached to the extracellular domain of the CAR for positioning of the CAR in a cell membrane. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one embodiment, the transmembrane domain is one that is associated with one of the other domains of the CAR e.g., in one embodiment, the transmembrane domain may be from the same protein that the primary signalling domain, costimulatory domain or the hinge region is derived from. In another embodiment, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one embodiment, the transmembrane domain is capable of homodimerization with another CAR on the cell surface of a CAR-expressing cell. In a different embodiment, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one embodiment the transmembrane domain is capable of signalling to the intracellular domain(s) whenever the CAR has bound to its target. A transmembrane domain of particular use in this invention may include at least the transmembrane region(s) of e.g., the alpha, beta or zeta chain of the T-cell receptor, TNFR2, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CDl la, LFA-1, ITGAM, CDl lb, ITGAX, CDl lc, ITGB1, CD29, ITGB2, CD18, LFA-1, ITGB7, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or a functional variant thereof.

In some instances, the transmembrane domain can be attached to the extracellular region of the CAR, e.g., the antigen-binding domain of the CAR, via a hinge, e.g., a hinge from a human protein. As used herein, the term “hinge region” refers to any suitable amino acid sequence positioned between the transmembrane domain and the antigen binding domain. Suitable hinge sequences are known in the art. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8 alpha hinge. In one embodiment, the hinge or spacer comprises an IgG4 hinge. In one embodiment, the hinge region comprises an IgD hinge. In another embodiment, the hinge comprises a CD8 alpha hinge region.

In one embodiment, the transmembrane domain may be recombinant, in which case it will comprise predominantly hydrophobic residues such as leucine and valine. In one embodiment a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one embodiment, the linker comprises the amino acid sequence of GGGGSGGGGS. In one embodiment, the hinge or spacer comprises a KIR2DS2 hinge.

### Intracellular Domain

The intracellular domain of the CAR is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular domain” refers to the portion of a CAR which transduces the effector function signal and directs the cell to perform a specialized function. While usually the entire intracellular domain can be employed, in many cases it is not necessary to use the entire chain. To the extent that a truncated portion of the intracellular domain is used, such truncated portion may be used in place of the intact chain as long as it transduces the effector function signal. The term intracellular domain is thus meant to include any truncated portion of the intracellular domain sufficient to transduce the effector function signal.

Examples of intracellular domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signalling sequences: those that initiate antigen-dependent primary activation through the TCR (referred to herein as a “primary signalling domain”) and those that act in an antigen-independent manner to provide a secondary or costimulatory signal (referred to herein as a “costimulatory domain”).

As used herein, the phrase “primary signalling domain” refers to a domain present in the intracellular domain of a CAR, which is capable of inducing, or inhbiting, antigen-dependent primary activation signalling in a cell expressing the CAR. For example, primary activiation signalling such as that initiated by, for instance, binding of a TCR/CD3 complex with an MHC molecule loaded with peptide, and which leads to mediation of a T cell response, such as proliferation, activation, differentiation, and the like. In this regard, a primary signalling domain regulates primary activation of the TCR complex either in a stimulatory way, or in an inhibitory way. Primary signalling domains that act in a stimulatory manner may contain signalling motifs which are known as immunoreceptor tyrosine-based activation motifs or ITAMs.

Examples of ITAM containing primary signalling domains that are of particular use in the invention include those of CD3 zeta, common FcR gamma (FCERIG), Fc gamma Rlla, FcR beta (Fc Epsilon Rib), CD3 gamma, CD3 delta, CD3 epsilon, CD79a, CD79b, DAP10, and DAP12, or a functional variant thereof. In one embodiment, a CAR of the invention comprises an intracellular signalling domain, e.g., a primary signalling domain of CD3-zeta, or a functional variant thereof. In one embodiment, a CAR of the invention comprises a primary signalling domain of CD3-zeta (e.g., SEQ ID NO:35).

In one embodiment, a primary signalling domain comprises a modified ITAM domain, e.g., a mutated ITAM domain which has altered (e.g., increased or decreased) activity as compared to the native ITAM domain. In one embodiment, a primary signalling domain comprises a modified ITAM-containing primary signalling domain, e.g., an optimized and/or truncated ITAM-containing primary signalling domain. In an embodiment, a primary signalling domain comprises one, two, three, four or more ITAM motifs.

The intracellular domain of the CAR can comprise the primary signalling domain by itself or it can be combined with any other desired intracellular signalling domain(s) useful in the context of a CAR of the invention. For example, in some embodiments, the intracellular domain of the CAR comprises a primary signalling domain and a costimulatory domain.

A “costimulatory domain” refers to a portion of the CAR which is derived from an intracellular domain of a costimulatory molecule and which is capable of providing a costimulatory signal in a cell expressing the CAR upon antigen binding. A costimulatory molecule is a cell surface molecule other than an antigen receptor or its ligands that is required for an efficient response of lymphocytes to an antigen. Examples of such molecules include TNFR2, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CAR-T cells in vitro and augments human T cell persistence and antitumour activity in vivo (Song et al., 2012). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDl lb, ITGAX, CDllc, ITGBl, CD29, ITGB2, CD18, LFA-1, ITGB7, TNFR2, TRANCE/RANKL, DNAMl (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, and CD19a.

The primary signalling domain and the costimulatory domain within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signalling sequence. In one embodiment, a glycine-serine doublet can be used as a suitable linker. In one embodiment, a single amino acid, e.g., an alanine, a glycine, can be used as a suitable linker.

In one embodiment, the intracellular domain is designed to comprise two or more, e.g., 2, 3, 4, 5, or more, costimulatory domains. In an embodiment, the two or more, e.g., 2, 3, 4, 5, or more, costimulatory domains, are separated by a linker molecule, e.g., a linker molecule described herein. In one embodiment, the intracellular domain comprises two costimulatory domains. In some embodiments, the linker molecule is a glycine residue. In some embodiments, the linker is an alanine residue.

In one embodiment, the intracellular domain is designed to comprise the signalling domain of CD3-zeta and the signalling domain of CD28. In one embodiment, the intracellular domain is designed to comprise the signalling domain of CD3-zeta, the signalling domain of CD28, and the signalling domain of 4-1BB, or a functional variant thereof. In one embodiment, the intracellular domain is designed to comprise the signalling domain of CD3-zeta, the signalling domain of CD28, and the signalling domain of CD27.

### Multispecific CARs

In some embodiments, the CAR is a multi-specific (e.g., a bispecific or a trispecific) CAR. Protocols for generating bispecific or heterodimeric binding molecules are known in the art; including but not limited to, for example, as described in U.S. Pat. No. 5,731,168; WO 09/089004, WO 06/106905 and WO 2010/129304; WO 07/110205; WO 08/119353, WO 2011/131746, and WO 2013/060867; U.S. Pat. Nos. 5,273,743; 5,534,254; 5,582,996; 5,591,828; 5,635,602; 5,637,481; 5,837,242; 5,837,821; 5,844,094; 5,864,019 and 5,869,620.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VHi) upstream of its VL (VLi) and the downstream antibody or antibody fragment (e.g., scFv) is arranged with its VL (VL2) upstream of its VH (V3/4), such that the overall bispecific antibody molecule has the arrangement VH1-VL1-VL2-VH2. In other embodiments, the upstream antibody or antibody fragment (e.g., scFv) is arranged with its VL (VLi) upstream of its VH (VHi) and the downstream antibody or antibody fragment (e.g., scFv) is arranged with its VH (VH2) upstream of its VL (VL2), such that the overall bispecific antibody molecule has the arrangement VL1-VH1-VH2-VL2. Optionally, a linker is disposed between the two antibodies or antibody fragments (e.g., scFvs), e.g., between VL1 and VL2 if the construct is arranged as VH1-VL1-VL2-VH2, or between VH2 and VH2 if the construct is arranged as VL1-VH1-VH2-VL2. The linker may be a linker as described herein. In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

### Regulatable CARs

A regulatable CAR (RCAR) is a CAR for which activity can be controlled is desirable to optimize the safety and efficacy of a CAR therapy. There are many ways CAR activities can be regulated. For example, inducible apoptosis using, e.g., a caspase fused to a dimerization domain (see, e.g., Di et al., 2011), can be used as a safety switch in the CAR therapy of the instant invention.

In embodiments, an RCAR can comprise a “multi switch,” A multi switch can comprise heterodimerization switch domains or homodimerization switch domains.

An embodiment provides RCARs wherein the antigen binding member is not tethered to the surface of the CAR cell. This allows a cell having an intracellular signalling member to be conveniently paired with one or more antigen-binding domains, without transforming the cell with a sequence that encodes the antigen binding member.

An embodiment provides RCARs having a configuration that allows switching of proliferation. In this embodiment, the RCAR comprises: 1) an intracellular signalling member comprising: optionally, a transmembrane domain or membrane tethering domain; one or more co-stimulatory signalling domain, e.g., selected from 41BB, CD28, CD27, ICOS, and OX40, and a switch domain; and 2) an antigen binding member comprising: an antigen-binding domain, a transmembrane domain, and a primary signalling domain, e.g., a CD3 zeta domain, wherein the antigen binding member does not comprise a switch domain, or does not comprise a switch domain that dimerizes with a switch domain on the intracellular signalling member. In an embodiment, the antigen binding member does not comprise a co-stimulatory signalling domain. In an embodiment, the intracellular signalling member comprises a switch domain from a homodimerization switch. In an embodiment, the intracellular signalling member comprises a first switch domain of a heterodimerization switch and the RCAR comprises a second intracellular signalling member which comprises a second switch domain of the heterodimerization switch. In such embodiments, the second intracellular signalling member comprises the same intracellular signalling domains as the intracellular signalling member. In an embodiment, the dimerization switch is intracellular. In an embodiment, the dimerization switch is extracellular.

In any of the RCAR configurations described here, the first and second switch domains comprise a FKBP-FRB based switch as described herein.

### Split CARs

In some embodiments, the CAR of the invention is a split CAR. The split CAR approach is described in more detail in publications WO2014/055442 and WO2014/055657.

### Determining Efficacy of a CAR

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of CARs of the present invention are described in further detail below.

Western blot analysis of CAR expression in primary T cells can be used to detect the presence of monomers and dimers (see, e.g., Milone et al., 2009). Very briefly, T cells (1:1 mixture of CD4+ and CD8+ T cells) expressing the CARs are expanded in vitro for more than 10 days followed by lysis and SDS-PAGE under reducing conditions. CARs containing the full length TCR-ζ cytoplasmic domain and the endogenous TCR-ζ chain are detected by western blotting using an antibody to the TCR-ζ chain. The same T cell subsets are used for SDS-PAGE analysis under non-reducing conditions to permit evaluation of covalent dimer formation.

In vitro expansion of CAR-T cells following antigen stimulation can be measured by flow cytometry. For example, a mixture of CD4+ and CD8+ T cells are stimulated with aCD3/aCD28 aAPCs followed by transduction with lentiviral vectors expressing GFP under the control of the promoters to be analysed. Exemplary promoters include the CMV IE gene, EF-la, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., 2009). Alternatively, a mixture of CD4+ and CD8+ T cells are stimulated with aCD3/aCD28 coated magnetic beads on day 0, and transduced with CAR on day 1 using a bicistronic lentiviral vector expressing CAR along with eGFP using a 2A ribosomal skipping sequence. Cultures are re-stimulated with either cells expressing the antigen, e.g., K562 cells (K562 expressing the antigen), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/ml. GFP expressing T cells are enumerated by flow cytometry using bead-based counting. Sustained CAR-T cell expansion in the absence of re-stimulation can also be measured (see, e.g., Milone et al., 2009). Briefly, mean T cell volume (fl) is measured on day 8 of culture using a Coulter Multisizer III particle counter, a Nexcelom Cellometer Vision or Millipore Scepter, following stimulation with aCD3/aCD28 coated magnetic beads on day 0, and transduction with the indicated CAR on day 1.

Animal models can also be used to measure a CAR's activity. For example, a xenograft model. Specific CAR-T cells to treat a primary human pre-B ALL in immunodeficient mice can be used (see, e.g., Milone et al., 2009). Very briefly, after establishment of ALL, mice are randomized as to treatment groups. Different numbers of CAR engineered T cells are coinjected at a 1:1 ratio into NOD-SCID-mice bearing B-ALL. The number of copies of the specific CAR vector in spleen DNA from mice is evaluated at various times following T cell injection. Animals are assessed for leukemia at weekly intervals. Peripheral blood blast cell counts are measured in mice that are injected with antigenic peptide for CAR-T cells or mock-transduced T cells. Survival curves for the groups are compared using the log-rank test. In addition, absolute peripheral blood CD4+ and CD8+ T cell counts 4 weeks following T cell injection in NOD-SCID-mice can also be analysed. Mice are injected with leukemic cells and 3 weeks later are injected with T cells engineered to express CAR by a bicistronic lentiviral vector that encodes the CAR linked to eGFP. T cells are normalized to 45-50% input GFP+ T cells by mixing with mock-transduced cells prior to injection, and confirmed by flow cytometry. Animals are assessed for leukemia at 1-week intervals. Survival curves for the CAR-T cell groups are compared using the log-rank test.

Assessment of cell proliferation and cytokine production has been previously described (e.g., Milone et al., 2009). Briefly, assessment of CAR-mediated proliferation is performed in microtiter plates by mixing washed T cells with K562 cells expressing an antigen (e.g., K19) or CD32 and CD137 (KT32-BBL) for a final T-cell:K562 ratio of 2:1. K562 cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8+ T cell expansion ex vivo. T cells are enumerated in cultures using CountBright™ fluorescent beads (Invitrogen, Carlsbad, CA) and flow cytometry as described by the manufacturer. CAR-T cells are identified by GFP expression using T cells that are engineered with eGFP-2A linked CAR-expressing lentiviral vectors. For CAR-T cells not expressing GFP, the CAR+ T cells are detected with biotinylated protein and a secondary avidin-PE conjugate. CD4+ and CD8+ expression on T cells are also simultaneously detected with specific monoclonal antibodies (BD Biosciences). Cytokine measurements (e.g., IFN gamma) are performed on supernatants collected 24 hours following re-stimulation using the human TH1/TH2 cytokine cytometric bead array kit (BD Biosciences, San Diego, CA) according the manufacturer's instructions. Fluorescence is assessed using a FACScalibur flow cytometer, and data is analysed according to the manufacturer's instructions.

Imaging technologies can be used to evaluate specific trafficking and proliferation of CARs in tumour-bearing animal models. Such assays have been described. for example, in Barrett et al., (2011).

Alternatively, therapeutic efficacy and specificity of a single injection of CAR-T cells in Nalm-6 xenograft model can be measured as the following: NSG mice are injected with Nalm-6 transduced to stably express firefly luciferase, followed by a single tail-vein injection of T cells electroporated with CARs of the present invention 7 days later. Animals are imaged at various time points post injection. For example, photon-density heat maps of firefly luciferase positive leukemia in representative mice at day 5 (2 days before treatment) and day 8 (24 hr post CAR+ PBLs) can be generated.

Other assays, including those described in the Example section herein as well as those that are known in the art can also be used to evaluate the CARs described herein.

### Nucleic Acids and Vectors

The present invention also provides vectors in which a nucleic acid construct encoding a binding protein or CAR of the invention is inserted. The expression of natural or synthetic nucleic acids encoding binding proteins or CARs is typically achieved by operably linking a nucleic acid encoding the binding protein or CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, and 5,589,466. In another embodiment. the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid.

In an embodiment, the vector comprising the nucleic acid encoding the desired protein or CAR of the invention is an adenoviral vector (A5/35). In another embodiment, the expression of nucleic acids encoding the protein or CAR can be accomplished using of transposons such as sleeping beauty, crisper, CAS9, and zinc finger nucleases (see e.g., June et al., 2009).

Further, the expression vector may be provided to a cell in the form of a viral vector. Viral vector technology is well known in the art and is described, for example, in Sambrook Molecular Cloning: A Laboratory Manual, volumes 1-4, Cold Spring Harbor Press, and in other virology and molecular biology manuals. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used. Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

### Methods of Making a Binding Protein- or CAR-Expressing Cell

Also provided herein are methods of making binding protein- or CAR-expressing cells by introducing a vector or nucleic acid encoding the protein or CAR into a cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, for example, Sambrook Molecular Cloning: A Laboratory Manual, volumes 1-4, Cold Spring Harbor Press). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

An exemplary non-viral delivery vehicle is a liposome. The use of lipid formulations is contemplated for the introduction of the nucleic acids into a host cell (in vitro, ex vivo or in vivo). In another embodiment, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid.

Regardless of the method used to introduce exogenous nucleic acids into a host cell or otherwise expose a cell to the inhibitor of the present invention, in order to confirm the presence of the recombinant DNA sequence in the host cell, a variety of assays may be performed Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Immune effector cells such as T cells may be activated and expanded generally using well known methods.

Generally, a population of immune effector cells e.g., T regulatory cell depleted cells, may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., 1998; Haanen et al., 1999; Garland et al., 1999).

Conditions appropriate for immune effector cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF, and TNF-a or any other additives for the growth of cells known to the skilled artisan.

### Cells Comprising CARs or Binding Proteins

In another aspect, the present invention provides a cell comprising the binding protein, the CAR, the nucleic acid, or the vector of the invention. Suitable cell types are described herein. In some embodiments, the cell is an immune effector cell. As used herein, the phrase “immune effector cell” refers to an immune cell which is capable of affecting or inducing an immune response upon recognition of an antigen. In some embodiments, the immune effector cell is a T cell or an NK cell. In some embodiments, the immune effector cell is a CD8+ T cell. In some embodiments, the cell is a mammalian cell. In some embodiments, the cell is a human cell.

In one embodiment, the cell described herein can further comprise a second (or more) CAR, e.g., a second CAR that includes a different antigen-binding domain, e.g., to the same target (e.g., a target described above) or a different target. In one embodiment, the second CAR includes an antigen-binding domain which binds to a target expressed on the same cancer cell type as the target of the first CAR.

In another embodiment, the cell comprising a binding protein or CAR described herein can further express another agent, e.g., an agent which enhances the activity of a CAR-expressing cell. For example, in one embodiment, the agent can be an agent which inhibits an inhibitory molecule. Inhibitory molecules, e.g., PDl, can, in some embodiments, decrease the ability of a CAR-expressing cell to mount an immune effector response. Examples of inhibitory molecules include PDl, PD-Ll, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta. In one embodiment, the agent which inhibits an inhibitory molecule, e.g., is a molecule described herein, e.g., an agent that comprises a first polypeptide, e.g., an inhibitory molecule, associated with a second polypeptide that provides a positive signal to the cell, e.g., an intracellular signalling domain described herein. In one embodiment, the agent comprises a first polypeptide, e.g., of an inhibitory molecule such as PDl, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 or TGF beta, or a fragment of any of these (e.g., at least a portion of an extracellular domain of any of these), and a second polypeptide which is an intracellular signalling domain described herein (e.g., comprising a costimulatory domain (e.g., 41BB, CD27 or CD28, e.g., as described herein, or a functional variant thereof) and/or a primary signalling domain (e.g., a CD3 zeta signalling domain described herein, or a functional variant thereof). In one embodiment, the agent comprises a first polypeptide of PD1 or a fragment thereof (e.g., at least a portion of an extracellular domain of PD1, or a functional variant thereof), and a second polypeptide of an intracellular signalling domain described herein (e.g., a CD28 signalling domain described herein and/or a CD3 zeta signalling domain described herein, or a functional variant thereof). PD1 is an inhibitory member of the CD28 family of receptors that also includes CD28, CTLA-4, ICOS, and BTLA. PD-1 is expressed on activated B cells, T cells and myeloid cells (Agata et al., 1996). Two ligands for PDl, PD-1 and PD-L2 have been shown to downregulate T cell activation upon binding to PDl (Freeman et al., 2000; Latchman et al., 2001; Carter et al., 2002). PD-L1 is abundant in human cancers (Dong et al., 2003; Blank et al., 2005; Konishi et al., 2004). Immune suppression can be reversed by inhibiting the local interaction of PDl with PD-L1.

In one embodiment, the agent comprises the extracellular domain (ECD) of an inhibitory molecule, e.g., Programmed Death 1 (PD1), fused to a transmembrane domain and intracellular signalling domains such as 41BB and CD3 zeta (also referred to herein as a PDl CAR). In one embodiment, the PDl CAR, when used in combinations with a XCAR described herein, improves the persistence of the T cell. In one embodiment, the CAR is a PD1 CAR comprising the extracellular domain of PD1.

In another embodiment, the present invention provides a population of CAR-expressing cells, e.g., CAR-T cells. In some embodiments, the population of CAR-expressing cells comprises a mixture of cells expressing different CARs. For example, in one embodiment, the population of CAR-T cells can include a first cell expressing a CAR having an antigen-binding domain, and a second cell expressing a CAR having a different antigen-binding domain, e.g., an antigen-binding domain to a different target, e.g., an antigen-binding domain to a target that differs from the target bound by the antigen-binding domain of the CAR expressed by the first cell. As another example, the population of CAR-expressing cells can include a first cell expressing a CAR that includes an antigen-binding domain to a first target, and a second cell expressing a CAR that includes an antigen-binding domain to a second target. In one embodiment, the population of CAR-expressing cells includes, e.g., a first cell expressing a CAR that includes a primary signalling domain, and a second cell expressing a CAR that includes a secondary signalling domain.

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not clicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

### Methods of Treatment

### Conditions to be Treated

The binding proteins, compositions, and CARs of the invention are useful, inter alia, for the treatment, prevention and/or amelioration of any disease or disorder associated with or mediated by EGFRvIII expression or activity, or treatable by blocking the interaction between EGFRvIII and an EGFR ligand or otherwise inhibiting EGFRvIII activity and/or signaling, and/or promoting receptor internalization and/or decreasing cell surface receptor number. For example, the binding proteins, compositions, and CARs of the present invention are useful for the treatment of tumors that express EGFRvIII and/or that respond to ligand-mediated signaling. The binding proteins, compositions, and CARs of the present invention may also be used to treat primary and/or metastatic tumors arising in the brain and meninges, oropharynx, lung and bronchial tree, gastrointestinal tract, male and female reproductive tract, muscle, bone, skin and appendages, connective tissue, spleen, immune system, blood forming cells and bone marrow, liver and urinary tract, and special sensory organs such as the eye. In certain embodiments, the binding proteins, compositions, and CARs of the invention are used to treat one or more of the following cancers: renal cell carcinoma, pancreatic carcinoma, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer, synovial sarcoma, thyroid cancer, breast cancer, or melanoma.

In the context of the methods of treatment described herein, the binding protein, composition, or CAR may be administered as a monotherapy (i.e., as the only therapeutic agent) or in combination with one or more additional therapeutic agents (examples of which are described elsewhere herein).

In an embodiment, the binding proteins of the present invention are also useful for treating cancers associated with overexpressed EGFR, preferably in cancers where EGFR is misfolded. In this case, the binding proteins are useful for treating glioma, pancreatic cancer, colorectal cancer, lung cancer, breast cancer, stomach cancer, renal cancer, cervical cancer, ovarian cancer, adenocarcinoma, bladder cancer, or head and neck cancer.

### Dosages and Timing of Administration

Suitable dosages of binding proteins, compositions, and cells of the present invention will vary depending on the the condition to be treated and/or the subject being treated. It is within the ability of a skilled physician to determine a suitable dosage

In some embodiments, a method of the present invention comprises administering a prophylactically or therapeutically effective amount of a binding protein, composition, or cells described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition.

As used herein, the term “prophylactically effective amount” shall be taken to mean a sufficient quantity of a binding protein, composition, or cells to prevent or inhibit or delay the onset of one or more detectable symptoms of a clinical condition. The skilled artisan will be aware that such an amount will vary depending on, for example, the specific binding protein(s) administered and/or the particular subject and/or the type or severity or level of condition and/or predisposition (genetic or otherwise) to the condition.

Administration of a binding protein, composition, or cells according to the methods of the present invention can be continuous or intermittent, depending, for example, on the recipient's physiological condition. whether the purpose of the administration is therapeutic or prophylactic, and other factors known to skilled practitioners. The administration may be essentially continuous over a preselected period of time or may be in a series of spaced doses, e.g., either during or after development of a condition.

As will be apparent to the skilled person a “reduction” in a symptom of a cancer in a subject will be comparative to another subject who also suffers from a cancer but who has not received treatment with a method described herein. This does not necessarily require a side-by-side comparison of two subjects. Rather population data can be relied upon. For example, a population of subjects suffering from a cancer who have not received treatment with a method described herein (optionally, a population of similar subjects to the treated subject, e.g., age, weight, race) are assessed and the mean values are compared to results of a subject or population of subjects treated with a method described herein.

### CAR Expressing Cells

The invention includes a type of cellular therapy where immune effector cells (e.g., T cells, NK cells) are genetically modified to express a CAR described herein and the CAR-expressing cell is infused to a recipient in need thereof The infused cell is able to kill diseased cells expressing the target of the CAR in the recipient. Unlike antibody therapies, CAR-modified immune effector cells (e.g., T cells, NK cells) are able to replicate in vivo resulting in long-term persistence that can lead to sustained tumour control. In various embodiments, the immune effector cells (e.g., T cells, NK cells) administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell or NK cell to the patient.

The invention also includes a type of cellular therapy where immune effector cells (e.g., T cells, NK cells) are modified, e.g., by in vitro transcribed RNA, to transiently express a chimeric antigen receptor (CAR) and the CAR T cell or NK cell is infused to a recipient in need thereof. The infused cell is able to kill tumour cells in the recipient. Thus, in various embodiments, the immune effector cells (e.g., T cells, NK cells) administered to the patient, is present for less than one month, e.g., three weeks, two weeks, one week, after administration of the T cell or NK cell to the patient. Without wishing to be bound by any particular theory, the anti-tumour immunity response elicited by the CAR-modified immune effector cells (e.g., T cells, NK cells) may be an active or a passive immune response, or alternatively may be due to a direct vs indirect immune response. In one embodiment, the CAR transduced immune effector cells (e.g., T cells, NK cells) exhibit specific proinflammatory cytokine secretion and potent cytolytic activity in response to human cancer cells expressing EGFRvIII.

Ex vivo procedures are well known in the art. Briefly, cells are isolated from a mammal (e.g., a human) and genetically modified (i.e., transduced or transfected in vitro) with a vector expressing a CAR described herein. The CAR-expressing cell can be administered to a mammalian recipient to provide a therapeutic benefit. The mammalian recipient may be a human and the CAR-expressing cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, can be applied to the cells of the present invention. Other suitable methods are known in the art. therefore the present invention is not limited to any particular method of ex vivo expansion of the cells. Briefly, ex vivo culture and expansion of immune effector cells (e.g., T cells, NK cells) comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

The CAR-expressing immune effector cells (e.g., T cells, NK cells) of the present invention may be administered either alone, or as a pharmaceutical composition in combination with diluents and/or with other components such as IL-2 or other cytokines or cell populations, as described herein. Immune effector cells may be administered either alone, or as a pharmaceutical composition in combination with diluents and/or with other components such as IL-2, IL-15, or other cytokines or cell populations. Briefly, pharmaceutical compositions may comprise immune effector cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration.

A pharmaceutical composition comprising the cells described herein may be administered at a dosage of 10⁴ to 10⁹ cells/kg body weight, such as 10⁵ to 10⁶ cells/kg body weight, including all integer values within those ranges. Cell compositions may also be administered multiple times at these dosages. The cells can be administered by using infusion techniques that are commonly known in immunotherapy (see, e.g., Rosenberg et al., 1988). The optimal dosage and treatment regime for a particular patient can readily be determined by one skilled in the art of medicine by monitoring the patient for signs of disease and adjusting the treatment accordingly.

### Combination Therapies

The binding proteins or CARs of the present invention may be co-formulated with and/or administered in combination with one or more additional therapeutically active component(s) of one or more chemotherapeutic agents.

### In Vitro and Diagnostic Uses

The binding proteins of the present invention may also be used to detect and/or measure EGFRvIII, or EGFRvIII-expressing cells in a sample, e.g., for diagnostic purposes. Alternativley, the binding proteins of the present invention may also be used to detect and/or measure EGFR, or EGFR-expressing cells in a sample, e.g., for diagnostic purposes, preferably where EGFR is misfolded. For example, a binding protein of the invention may be used to diagnose a condition or disease characterized by expression of EGFRvIII. Exemplary diagnostic assays for EGFRvIII or EGFR may comprise, e.g., contacting a sample, obtained from a patient, with a binding protein of the invention, wherein the binding protein is labeled with a detectable label or reporter molecule. Alternatively, an unlabeled binding protein of the invention can be used in diagnostic applications in combination with a secondary protein which is itself detectably labeled. The detectable label or reporter molecule can be a radioisotope, such as ³H, ¹⁴C, ³²S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure EGFRvIII in a sample include enzyme-linked immunosorbent assay (ELISA), radioimmunoassay (RIA), and fluorescence-activated cell sorting (FACS).

Samples that can be used in EGFRvIII or EGFR diagnostic assays according to the present invention include any tissue or fluid sample obtainable from a patient which contains detectable quantities of EGFRvIII or EGFR protein, or fragments thereof, under normal or pathological conditions. Generally. levels of EGFRvIII or EGFR in a particular sample obtained from a healthy patient (e.g., a patient not afflicted with a disease or condition associated with EGFRvIII expression) will be measured to initially establish a baseline, or standard, level of EGFRvIII or EGFR. This baseline level of EGFRvIII or EGFR can then be compared against the levels of EGFRvIII or EGFR measured in samples obtained from individuals suspected of having a EGFRvIII or EGFR associated disease or condition.

### EXAMPLES

### Example 1—Materials and Methods

Generation of Anti-EGFRvIII scFvs

Recombinant soluble EGFRvIII protein (Cat #EGI-H52H4) and the wild-type EGFR (Cat #EGR-H5222) consisting of the extracellular domains, and without the transmembrane or internal domains, were purchased from ACRO Biosystems. Recombinant EGFRvIII was labelled with the chemical fluorophores DyLight 405 NHS ester (ThermoFisher, Cat #46400) and ATTO 488 NHS ester (ATTO-TEC, Cat #AD 488-31) according to the manufacturer's instructions. Recombinant EGFR was labelled with DyLight 405 NHS ester.

Affinity Biosciences' Retained Display (ReD) protein display platform (WO2011075761A1 and WO2013000023A1) and RUBY libraries (WO2013023251A1) were used to screen for scFv antibodies with human germline scaffolds that would selectively target the EGFRvIII form over the wild-type EGFR protein Following two rounds of panning of the RUBY bacteriophage libraries using EGFRvIII protein bound to Dynabeads (ThermoFisher, Cat #65002), the panning output was switched to the cell-display modality of ReD and treated as described in patent WO2013000023A1. The detergent-permeabilised cells presenting scFv bound to the capsid were labelled with EGFRvIII-ATTO488 and clones that bound to the target selected by FACS. FACS output was lysed and the bacteriophage infected and clones recovered on LB agar plates. A second and third round of FACS was performed in a similar manner, using EGFRvIII labelled with Dy405 for the second round and ATTO488 for the third round of FACS. Two rounds of counter-screening FACS were then conducted, first using EGFRvIII labelled with Dy405 and EGFR with ATTO488 to gate for clones that selectively bound the Dy405-labelled EGFRvIII. The second FACS counter-screen reversed the dye-labelling and selected for clones binding to the ATTO488-labelled target. The output clones from the second counterscreen were sequenced and unique scFvs were cloned into an expression vector that was produced the scFv protein as a His6- and AviTag-fusion to aid with purification and characterization.

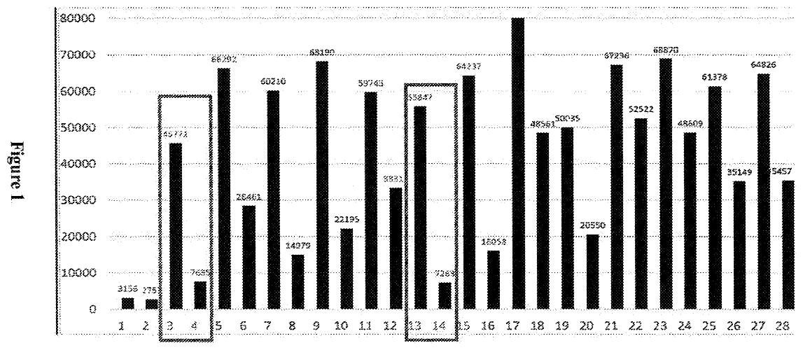

Sequencing of the EGFRvIII screen output yielded 13 clones that preferentially bound EGFRvIII over the wild-type EGFR extracellular domains. Selectivity was demonstrated by an assay where biotinylated scFv was first bound to streptavidin Dynabeads, which were then washed and blocked with free biotin, before binding to soluble EGFRvIII or EGFR that was labelled with ATTO488. An unrelated biotinylated scFv was used as a control to establish the baseline of binding and fluorescence. FIG. 1 demonstrates the relative binding of scFv clones to either EGFRvIII or EGFR. Two clones 70 and 205, renamed GCT01 (VH/VL SEQ ID NOs: 3/4) and GCT02 (VH/VL SEQ ID NOs: 5/6) showed negligible binding to the EGFR wild-type while maintaining strong binding to the EGFRvIII isoform. GCT01 was notable as it was related in its VH CDR3 to 10 other unique clones, suggesting a conserved binding epitope in EGFRvIII. All 10 clones in this clade used the same VL germline scaffold (IGLV3-1) but their VL CDR3s showed only weak sequence conservation (the central Gly was a feature of the library build using the IGLV3-1 scaffod), suggesting that the VH CDR3 made the crucial binding determinants. However, the sublties of the GCT01 sequence compared to the other clones of the clade conferred that clone with a higher discrimination of EGFRvIII against EGFR.

### Generation, Purification and Activation of Mouse T Cells

Lymph nodes from 6-8 week old C57BL/6 mice were harvested and filtered through 0.7 μm nylon filters. Cells were washed with PBS, centrifuged (1500 RPM, 5 minutes). Cells resuspended at 1×10⁸ cells/ml and CD4 or CD8 T cells were positively selected using a EasySep™ Positive selection kit, according to manufacturers instructions. Primary T cells were activated with mouse CD3z/CD28 dynabeads® and incubated at 37° C., 5% CO₂ overnight.

### Detecting EGFRvIII Expression on Target Cells

Target cells were labelled with GCT01, GCT02 scFv for 30 minutes on ice. Cells were labelled for 30 minutes with Streptavidin-PE. Samples were analysed by flow cytometry and cells were gated on PE positive cells.

### Surface Plasmon Resonance (SPR)

SPR experiments were performed using ProteOn XPR36 instrument (Bio-Rad) at 25° C. in PBS containing 0.05% Tween (PBS-T). Streptavidin diluted into 10 mM sodium acetate (pH 4.0), and 300 response units (RU) immobilized to three flow cells of GLC sensorchip by amine coupling. Biotinylated GCT01, GCT02 and GFP scFv chains (200-400 RU) were captured onto separate flow cells of ProteOn GLC streptavidin-coupled sensorchip. A separate flow cell containing immobilized streptavidin without coupled scFv served as control channel.

Recombinant EGFR Cat EGR-H5222 Lot: R102-616S1-A3 and EGFRvIII (protein Cat EGI-H52H4 Lot: 2144-65RS1-BG was obtained from Acrobiosystems and reconstituted to 1 ug/ul in PBS before dilution into PBS-T and injection over test and control surfaces at the concentrations indicated, (flow rate 30 μl/min). GFP protein was injected over chips to serve as negative control for non-specific scFv binding. A 10 mM glycine HCl buffer (pH 3) used to strip injected protein from chip between injections. Results from at least two independent injections were analyzed. After subtraction of data from control cells, interactions analyzed with ProteOn Manager software (version 2.1). KD values were derived from kinetic fit analysis using 1:1 Langmuir binding model.

### Generation of EGFRvIII CAR Expressing Cells

A MYC tag was inserted into the scFv region in order to confirm cell surface expression. Gene blocks were ordered and subcloned, using Gibson cloning, into the pMSCV plasmid backbone and fully sequence verified.

CAR plasmids were combined with retroviral envelope and packaging plasmids and transfected into HEK293T cells using FuGENE-6 (Promega), according to manufacturers instructions. Retronectin (32 ug/mL) was coated in 12-well plates at 4° C. overnight and then washed prior to transduction. HEK293T viral supernatant was filtered and centrifuged with the primary activated T cells and incubated for >4 hrs, before centrifugation for 1 hr at 1000 G and incubated at 37° C., 5% CO₂ overnight. The lymph nodes of 6-8 week old C57BL/6 mice were harvested, and the CD4⁺ and CD8⁺ T cell population were positively selected, before activation with CD35ζ/CD28 Dynabeads. Retrovirus in the supernatant was collected at 24 hrs and used to transduce the activated T cells. 24 hours after transduction, cells were analysed by flow cytometry to evaluate transduction efficiency by mCherry expression and cell surface Myc labelling.

### Generating the Non-Signalling EGFRvIII Expressing Cell Lines

Lentivirus containing non-signalling EGFRvIII (SEQ ID NO:40) was made using HEK293T cells and transduced into tumour cell lines. Tumour cell lines were stained using in house anti-EGFRvIII binders and EGFRvIII positive cells were FACS sorted, expanded and used in assays.

### Chromium-Release Assay

1×10⁶ target tumour cells were chromium (⁵¹Cr) labelled for 1 hour (37° C.) before washing and co-culturing with CAR-T cells at 10:1 effector cell to target cell ratio. Minimum killing was measured by target cells alone and maximum killing measured by target cells with 10% Triton X-100. Target and effector cells were co-incubated in 96-well plate (triplicate) for 24 hours at 37° C. 5% CO₂. Lysis was measured in viral supernatant using a Genesys GeneII scintillation counter.

### In Vivo Testing of CAR-T Cells in Mice Bearing Intracranial Tumours

Mice were administered 10 mg/kg prophalactic Baytril (Enrofloxacin) antibiotic 1 hr prior to surgery. General anaesthetic (GA) Ketamine to Xylazine (20,000 mg/Kg (ie. 0.4 g (400 μl) per 20 g Mouse combined) was administered i.p. and a subcutaneous injection of 5 mg/kg Carprofen analgesic was administered. Once the mouse was secured onto a sterotactic frame the injection site was identified as 2 mm lateral to the bregma at the coronal suture to a depth of 3 mm. A sagittal incision was made in the scalp with a sterile blade over the determined target. A small right frontal burr hole 3 mm was fashioned using an electric burr. 10⁵ tumour cells were injected 3 mm deep into the brain under stereotactic guidance using a 26-gauge needle attached to a Hamilton 10 μl microlitre syringe via fine plastic tubing driven by World Precision Instruments™. Aladdin™ AL-1000 electronic programmable syringe pump was used to dispense the tumour cells at 5 μl over 3 minutes. The injection site is closed with bone wax and the scalp was closed with vicryl dissolvable braided sutures using an interrupted suturing technique. The mice were administered with 10 mg/kg Baytril subcutaneous injection for 3 days post-surgery.

Tumour growth was monitored using the IVIS system (IVIS® Lumina III Series Hardware with Living Image® software (Perkin Elmer)). Briefly, mice were injected intra-peritoneally with 200 μL of Promega D-luciferin (in PBS) using a 27G insulin syringe. After approximately 5-10 minutes to allow the luciferin to circulate the body, mice were anaesthetized using Isoflurane inhalant (at a level between 4% and maintained at 2%) and transferred to the IVIS imaging system for imaging.

Following positive IVIS imaging of tumour engraftment performed 5-8 days post tumour inoculation mice were randomly assigned to groups and intraveneously administered between 2e6-10e6 CD4 and CD8 CAR T cells. Mice were then imaged using the IVIS® Lumina weekly to monitor tumour cell growth.

### Example 2—High Affinity Anti-EGFRvIII scFvs

Two high affinity anti-EGFRvIII scFvs, which bound specifically to EGFRvIII compared to wild type EGFR, were produced using a phage display screen at Myrio Therapeutics. Victoria, as described in Example 1. The two high affinity anti-EGFRvIII scFvs were termed “GCT01” and “GCT02”. Their amino acid sequences are shown below.

GCT01 VH amino acid sequence (CDRs underlined)
SEQ ID NO: 3
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
EQWEGDYFSAFDIWGQGTMVTVSS
GCT01 VL amino acid sequence (CDRs underlined)
SEQ ID NO: 4
QSVLTQPPSVSVSPGQTASITCSGDKLGDKYACWYQQKPGQSPVLVIYQ
DSKRPSGIPERFSGSNSGNTATLTISGTQAMDEADYYCQAWDSDGKATV
VFGTGTKVTVSS
GCT02 VH amino acid sequence (CDRs underlined)
SEQ ID NO: 5
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
GGENTFESHPVAFDIWGQGTMVTVSS
GCT02 VL amino acid sequence (CDRs underlined)
SEQ ID NO: 6
NFMLTQPHSVSESPGKTVTISCTRSSGSIASNYVQWYQQRPGSAPTTVI
YEDNQRPSGVPDRFSGSIDSSSNSASLTISGLKTEDEADYYCQSYDLAA
DVRVVFGTGTKVTVSS

### Example 3—Binding to EGFRvIII

Surface plasmon resonance (SPR) experiments were performed using a ProteOn XPR36 instrument (Bio-Rad) to determine the binding characteristics of GCT01 and GCT02 to EGFRvIII. Biotinylated GCT01, GCT02 and GFP scFv chains (200-400 RU) were captured onto separate streptavidin-coupled sensorchips. A separate flow cell containing immobilized streptavidin without coupled scFv served as control channel.

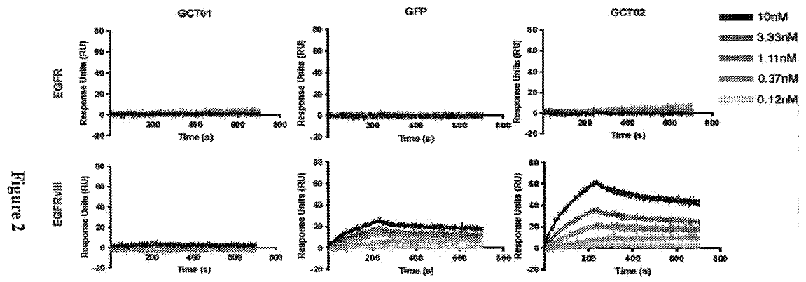

FIG. 2 shows GCT01 and GCT02 scFvs are specific for the EGFRvIII protein over EGFR Wild Type protein. Both GCT01 and GCT02 scFv clones bound specifically to recombinant EGFRvIII protein over EGFR (wild type) recombinant protein as determined by Surface plasmon resonance (SPR). GFP protein was used as a negative control. Recombinant EGFR and EGFRvIII (extracellular domain only) protein were reconstituted to 1 ug/ul in PBS before dilution into PBS-T and injection over test and control surfaces at concentrations between 0.1 nM and 10 nM. As shown in FIG. 2, both GCT01 and GCT02 bound to EGFRvIII and only very limited binding was detected for wild-type EGFR. Dissociation constant (KD) values were derived from kinetic fit analysis using a1:1 Langmuir binding model and were calculated to be 4.57×10-11 M for GCT01 and 3.27×10-10 M for GCT02. The affinities of GCT01 and GCT02 for EGFRvIII were several orders of magnitude greater than the affinities previously reported for a humanized scFv called “2173” or “N2173” (1.01×10-7 M) and a murine scFv called “3C10” (2.58×10-8 M) in Johnson et al. (2015).

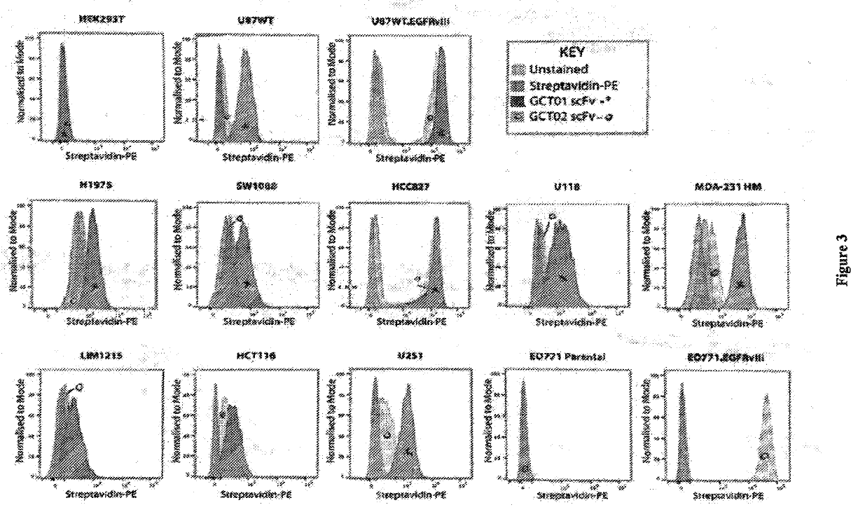

Flow cytometry was also performed to determine GCT01 and GCT02's ability to bind to EGFR expressed on the surface of cells. Importantly, both GCT01 and GCT02 scFv clones bound to U87 EGFRvIII cells specifically, with only GCT01 displaying binding to U87 wild-type cells. This confirmed that GCT01 and GCT02 bind specifically to both recombinant soluble EGFRvIII protein (FIG. 2) and also to EGFRvIII expressed on target cell membranes (FIG. 3).

Interestingly, FIG. 3 also shows that GCT01 demonstrates potential binding to misfolded EGFR as well as EGFRvIII, as detected on HCC827 (lung adenocarcinoma cells), U251 (human glioblastoma cells) and low level binding to HCT116 (human colorectal carcinoma cells).

### Example 4—EGFRvIII CAR Activity

The GCT01 and GCT02 scFv genes were codon optimized for expression in human cells and were cloned into a retroviral vector for CAR T cell production. The codon optimized nucleotide sequences for both scFvs are provided in SEQ ID NOs: 29 to 34.

The CAR constructs each comprised the GCT01 or GCT02 scFv, a CD8 hinge, a CD28 transmembrane domain, and an intracellular domain comprising a CD28 costimulatory domain and a CD3zeta primary signalling domain. The CARs were also expressed with an N-terminal leader sequence for localization to the T cell membrane and a Myc tag for confirming expression of the CARs. The amino acid sequences of the complete CARs are provided in SEQ ID NOs:25 and 26.

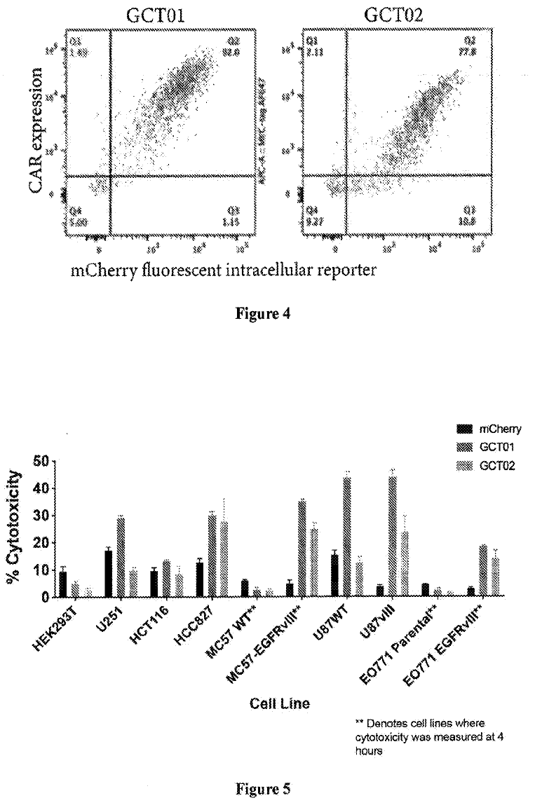

Primary T cells were transduced with the retroviral vectors as described in Example 1 and the transduction efficiency was measured by flow cytometry. FIG. 4 shows GCT01 and GCT02 Chimeric Antigen Receptors are expressed on the T cell surface. Cell surface CAR expression as determined by flow cytometry, using a Myc-tag located underneath the scFv. GCT01 (left) shows more concordant expression with the intracellular fluorophore mCherry (used as a surrogate measure of transduction efficiency) than GCT02 (right). Both constructs show very high expression of mCherry. These data are representative of five experiments. FIG. 4 shows that greater than 70% of primary T cells in any given were shown to be positive for CAR expression for both GCT01 and GCT02.

A chromium release assay was used to determine if the GCT01 and GCT02 CAR-expressing T cells had cytotoxic activity against cells expressing EGFR. Chromium release assays measure target cell death by quantification of radioactive chromium (⁵¹Cr) released from target cells, into the assay supernatant.

Cytotoxicity of the EGFRvIII CAR-T cells against a variety of cell lines was tested and the results are shown in FIG. 5. mCherry empty vector T cells were used as a negative control. The effector to target ratio was 10:1. Cytotoxicity was determined by chromium release, measured at 24 hours of coincubation (for cell lines marked with “***” in FIG. 5 cytotoxicity was measured after 4 hours of coincubation). The background level of non-specific target cell death was ˜10%. As expected, there was no specific target cell killing by any CART cells of HEK293T, MC57 WT or E0771 parental cell lines not expressing EGFRvIII. As a positive control for cytotoxicity, MC57, U87 and E0771 target cells were transduced with a non-signaling EGFRvIII construct in order to achieve high EGFRvIII cell surface expression. In these cell lines the GCT01 and GCT02 CAR T cells induced EGFRvIII-specific target cell death.

There was also cytotoxicity of U87 WT, U251 and HCT116 cells induced by the GCT01 CD8⁺ CART cells, which correlated with the binding of the GCT01 scFv clone to these cell lines. Genomic amplification of EGFR has been observed in 40-70% of glioblastomas and can lead to overexpression and misfolding at the cell surface. EGFR has been shown to be expressed in U87 cells, indicating that GCT01 binds to misfolded EGFR on cancer cells as well as EGFRvIII.

### Example 5—GCT01 and GCT02 CAR-T Cells Induce Tumour Regression of Intracranial Glioma Tumours

Firefly luciferase labelled U87-EGFRvIII tumour cells were grown intracranially in immunocompromised NSG mice for one week. A 1:1 mixture of CD4 and CD8 T cells (5×10⁶ cells) expressing either an empty mCherry labelled CAR, a GCT01-CAR, or a GCT02-CAR were then administered in a single i.v. injection.

FIG. 7 shows GCT01 and GCT02 CAR T cells induce rapid and complete clearance of in vivo glioblastoma brain tumours. The data shows that in week 3, little to no luciferase activity was detected in the GCT01-CAR and GCT02-CAR treated mice, indicating that cells expressing these receptors were capable of inducing tumour regression in this model of glioma.

### Example 6—GCT01 CAR-T Cells Induce Tumour Regression in a Model of Lung Adenocarcinoma

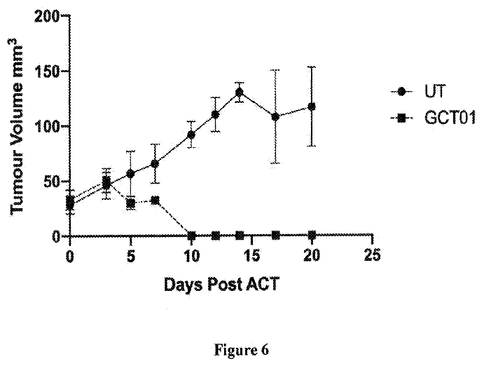

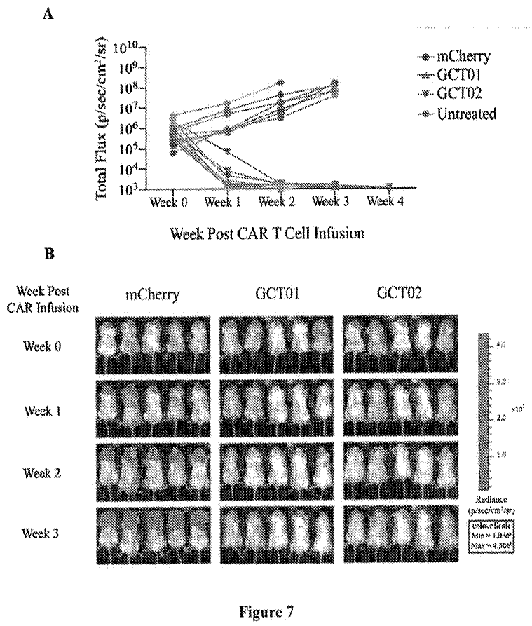

To further characterise the efficacy of GCT01 CAR T cells in vivo a sub-cutaneous model of lung adenocarcinoma was used. Briefly. 2e6 HCC827 cells were injected sub cutaneously into female NSG mice. 5-8 days later 2-10e6 CD4 and CD8 GCT01 CAR T cells were adoptively transferred into the mice intraveneously. Tumour growth was measured 3 times weekly using calipers. All tumour bearing NSG mice treated with GCT01 CAR T cells had their tumours regress within 14 days post adoptive transfer (FIG. 6).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Agata et al. (1996) Int. Immunol 8:765-75
Aggen et al. (2012) Gene Ther. 19:365-74
Al-Lazikani et al. (1997) J Mol Biol 273:927-948
Altschul et al. (1977) Nuc. Acids Res. 25:3389-3402
Altschul et al. (1990) J. Mol. Biol. 215:403-410
Bacuerle et al. (2008) Drugs of the Future 33:137-147
Bargou et al. (2008) Science 321:974-977
Barrett et al. (2011) Human Gene Therapy 22:1575-1586
Berg et al. (1998) Transplant Proc. 30:3975-3977
Bird et al. (1988) Science 242:423-426
Blank et al. (2005) cancer Immunol. Immunother 54:307-314
Brent et al. (2003) Current Protocols in Molecular Biology
Carter et al. (2002) Eur J Immunol 32:634-43
Chothia et al. (1987) J. Mol. Biol. 196:901
Chowdhury (2008) Methods Mol. Biol. 207:179-196
Cunningham (1989) Science 244:1081-1085
Di et al. (2011) N Egnl. J. Med 365:1673-1683
Dong et al. (2003) J Mol Med 81:281-7
Freeman et al. (2000) J Exp Med 192:1027-34
Garland et al. (1999) J. Immunol Meth. 227:53-63
Haanen et al. (1999) J. Exp. Med. 190:1319-1328
Hamers-Casterman et al. (1993) Nature 363:446-448
Hollinger et al. (1993) PNAS 90:6444-6448
Honnegher and Plükthun (2001) J. Mol. Biol., 309:657-670
Huston et al. (1988) PNAS 85:5879-5883
Johnson et al. (2015) Sci Transl Med. 7:275
June et al. (2009) Nature Reviews Immunology 9:704-716
Konishi et al. (2004) Clin cancer Res 10:5094
Lanzavecchia et al. (1987) Eur. J. Immunol. 17:105
Latchman et al. (2001) Nat Immunol 2:261-8
Lefranc et al. (2003) Devel. And Compar. Immunol., 27:55-77
Meyers and Miller (1988) Comput. Appl. Biosci. 4:11-17
Milone et al. (2009) Molecular Therapy 17:1453-1464
Needleman and Wunsch (1970) J. Mol. Biol. 48:443
Pearson and Lipman (1988) Proc. Nat'l. Acad. Sci. USA 85:2444
Rosenberg et al. (1988) New Eng. J. of Med 319:1676
Smith and Waterman (1970) Adv. Appl. Math. 2:482c
Song et al. (2012) Blood 119:696-706
Streltsov (2005) Protein Sci. 14:2901-2909
Willemsen et al. (2000) Gene Therapy 7:1369-1377
Zhang et al. (2004) cancer Gene Ther 11:487-496

1. A human epidermal growth factor receptor variant III (EGFRvIII) binding protein comprising an antigen binding domain which binds to EGFRvIII, wherein the binding protein competitively inhibits binding of EGFRvIII to an antibody comprising:
a heavy chain variable region (VH) comprising a CDR1, a CDR2, and a CDR3 comprising the sequences set forth in SEQ ID NOs: 13, 14, and 15 respectively; and a light chain variable region (VL) comprising a CDR1, a CDR2, and a CDR3 comprising the sequences set forth in SEQ ID NOs: 16, 17, and 18 respectively; or
a heavy chain variable region (VH) comprising a CDR1, a CDR2, and a CDR3 comprising the sequences set forth in SEQ ID NOs: 19, 20, and 21 respectively; and a light chain variable region (VL) comprising a CDR1, a CDR2, and a CDR3 comprising the sequences set forth in SEQ ID NOs: 22, 23, and 24 respectively; or
a VH comprising an amino acid sequence set forth in SEQ ID NO:3 and a VL comprising an amino acid sequence set forth in SEQ ID NO:4; or
a VH comprising an amino acid sequence set forth in SEQ ID NO:5 and a VL comprising an amino acid sequence set forth in SEQ ID NO:6.

2. (canceled)

3. The EGFRvIII binding protein of claim 1, wherein the binding protein binds to human EGFRvIII with an affinity of at least about 1 nM.

4. The EGFRvIII binding protein of claim 1, wherein the binding protein comprises:
a heavy chain variable region (VH) comprising a CDR1, a CDR2, and a CDR3 comprising the sequences set forth in SEQ ID NOs: 7, 8, and 9 respectively; and/or a light chain variable region (VL) comprising a CDR1, a CDR2, and a CDR3 comprising the sequences set forth in SEQ ID NOs: 10, 11, and 12 respectively; or
a heavy chain variable region (VH) comprising a CDR1, a CDR2, and a CDR3 comprising the sequences set forth in SEQ ID NOs: 13, 14, and 15 respectively; and/or a light chain variable region (VL) comprising a CDR1, a CDR2, and a CDR3 comprising the sequences set forth in SEQ ID NOs: 16, 17, and 18 respectively; or
a VH comprising a CDR1, CDR2, and a CDR3 comprising the amino acid sequences set forth in SEQ ID NOs: 13, 14, and 15 respectively; and/or a VL comprising a CDR1, CDR2, and a CDR3 comprising the amino acid sequences set forth in SEQ ID NOs: 16, 17, and 18 respectively; or
a VH comprising a CDR1, CDR2, and a CDR3 comprising the amino acid sequences set forth in SEQ ID NOs: 19, 20, and 21 respectively; and/or a VL comprising a CDR1, CDR2, and a CDR3 comprising the amino acid sequences set forth in SEQ ID NOs: 22, 23, and 24 respectively.

5. The EGFRvIII binding protein of claim 1, wherein the binding protein comprises:
a VH comprising a CDR1 comprising an amino acid sequence having no more than 2 amino acid substitutions relative to SEQ ID NO: 13, a CDR2 comprising an amino acid sequence having no more than 4 amino acid substitutions relative to SEQ ID NO: 14, and a CDR3 comprising an amino acid sequence having no more than 3 amino acid substitutions relative to SEQ ID NO: 15, and/or
a VL comprising a CDR1 comprising an amino acid sequence having no more than 3 amino acid substitutions relative to SEQ ID NO: 16, a CDR2 comprising an amino acid sequence having no more than 2 amino acid substitutions relative to SEQ ID NO: 17, and a CDR3 comprising an amino acid sequence having no more than 3 amino acid substitutions relative to SEQ ID NO: 18; or
a VH comprising a CDR1 comprising an amino acid sequence having no more than 2 amino acid substitutions relative to SEQ ID NO: 19, a CDR2 comprising an amino acid sequence having no more than 4 amino acid substitutions relative to SEQ ID NO:20, and a CDR3 comprising an amino acid sequence having no more than 3 amino acid substitutions relative to SEQ ID NO:21, and/or
a VL comprising a CDR1 comprising an amino acid sequence having no more than 3 amino acid substitutions relative to SEQ ID NO:22, a CDR2 comprising an amino acid sequence having no more than 2 amino acid substitutions relative to SEQ ID NO:23, and a CDR3 comprising an amino acid sequence having no more than 3 amino acid substitutions relative to SEQ ID NO:24.

6. (canceled)

7. The EGFRvIII binding protein of claim 1, wherein the binding protein comprises:
a VH comprising an amino acid sequence at least about 70% identical, at least about 80% identical, at least about 90% identical, or at least about 95% identical to the sequence set forth in SEQ ID NO: 3, and/or a VL comprising an amino acid sequence at least about 70% identical, at least about 80% identical, at least about 90% identical, or at least about 95% identical to the sequence set forth in SEQ ID NO:4; or
a VH comprising an amino acid sequence at least about 70% identical, at least about 80% identical, at least about 90% identical, or at least about 95% identical to the sequence set forth in SEQ ID NO: 5, and/or a VL comprising an amino acid sequence at least about 70% identical, at least about 80% identical, at least about 90% identical, or at least about 95% identical to the sequence set forth in SEQ ID NO:6; or
a heavy chain variable region (VH) comprising an amino acid sequence set forth in SEQ ID NO:3 and/or a light chain variable region (VL) comprising an amino acid sequence set forth in SEQ ID NO: 4; or
a heavy chain variable region (VH) comprising an amino acid sequence set forth in SEQ ID NO:5 and/or a light chain variable region (VL) comprising an amino acid sequence set forth in SEQ ID NO:6.

8. (canceled)

9. The EGFRvIII binding protein of claim 1, comprising a VH and a VL, wherein the VH and VL bind to form a Fv comprising an antigen binding site; or wherein the VH and the VL are in a single polypeptide chain; or wherein the VL and VH are in separate polypeptide chains.

10. (canceled)

11. The EGFRvIII binding protein of claim 9, wherein the binding protein comprises: (i) a single chain Fv fragment (scFv); (ii) a dimeric scFv (di-scFv); or at least one of (i) and/or (ii) linked to a constant region of an antibody, a fragment crystallizable (Fc) region or a heavy chain constant domain (CH) 2 and/or CH3; or
wherein the binding protein comprises: (iii) a diabody; (iv) a triabody; (v) a tetrabody; (vi) a Fab; (vii) a F(ab′)2; (viii)a Fv; or at least one of (iii) to (viii) linked to a constant region of an antibody, an Fc region or a CH2 and/or CH3.

12. The EGFRvIII binding protein of claim 1, wherein the binding protein is a bi-specific T cell engager (BiTE).

13. (canceled)

14. (canceled)

15. The EGFRvIII binding protein of claim 1, wherein the binding protein comprises a Fc region; wherein the binding protein is an antibody; and/or wherein the binding protein is conjugated to another compound.

16. (canceled)

17. (canceled)

18. A composition comprising the binding protein of claim 1, and a pharmaceutically acceptable carrier.

19. A chimeric antigen receptor (CAR) comprising an antigen-binding domain of claim 1, a transmembrane domain, and an intracellular domain.

20. The CAR of claim 19, wherein the intracellular domain comprises a primary signalling domain and a costimulatory domain.

21. The CAR of claim 20, wherein the primary signalling domain comprises a CD3 zeta, CD3 gamma, CD3 delta, CD3 epsilon, common FcR gamma (FCER1G), FcRbeta (Fc Epsilon Rib), CD79a, CD79b, Fcgamma Rlla, DAP 10, or DAP 12 primary signalling domain.

22. (canceled)

23. The CAR of claim 20, wherein the costimulatory domain comprises a CD28, 4-1BB (CD137), 0X40, CD27, CD30, CD40, CD134, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFl), CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD 103, ITGAL, CDlla, LFA-1, ITGAM, CDllb, ITGAX, CDllc, ITGB1, CD29, ITGB2, CD 18, LFA-1, ITGB7, TRAN CE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, or TNFR2 costimulatory domain.

24. (canceled)

25. The CAR of claim 19, wherein the transmembrane domain comprises a CD28, CD3 epsilon, CD3 zeta, CD45, CD4, CD5, CD8, CD9, CD16, CD22, CD33, CD37, CD64, CD80, CD86, CD134, CD137, CD154, KIRDS2, 0X40, CD2, CD27, LFA-1 (CD1 la, CD18), ICOS (CD278), 4-1BB (CD137), GITR, CD40, BAFFR, HVEM (LIGHTR), SLAMF7, NKp80 (KLRFl), CD160, CD19, IL2R beta, IL2R gamma, IL7Ra, ITGA1, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD 18, LFA-1, ITGB7, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD 150, IPO-3), BLAME (SLAMF8), SELPLG (CD 162), LTBR, PAG/Cbp, NKp44, NKp30, NKp46, NKG2D, NKG2C, or TNFR2 transmembrane domain, or the alpha, beta or zeta chain of the T-cell receptor.

26. (canceled)

27. The CAR of claim 19, wherein the antigen-binding domain is connected to the transmembrane domain by a hinge region.

28. The CAR of claim 27, wherein the hinge region comprises a CD8 hinge, an IgG hinge, an IgD hinge, a CD28 hinge, a KIR2DS2 hinge, or a glycine-serine linker.

29. The CAR of claim 19, wherein the CAR comprises an amino acid sequence which is at least about 70%, at least about 80%, at least about 90%, at least about 95% identical or 100% identical to the sequence set forth in SEQ ID NO:25 or SEQ ID NO: 26.

30. A nucleic acid encoding the binding protein of claim 1 or a CAR comprising an antigen binding domain of claim 1, optionally wherein the nucleic acid comprises a nucleotide sequence that is codon optimized for expression in human cells.

31. (canceled)

32. The nucleic acid of claim 30, wherein the nucleic acid comprises a nucleotide sequence which is at least about 70%, at least about 80%, at least about 90%, at least about 95% identical or 100% identical to any one or more of the sequences provided in SEQ ID NOs: 29, 30, 31, 32, 33 or 34.

33. A vector comprising the nucleic acid of claim 30, wherein the vector is a DNA vector, an RNA vector, a plasmid, a lentivirus vector, adenovirus or adeno-associated virus vector, or a retrovirus vector.

34. (canceled)

35. A cell comprising the binding protein of claim 1, the a CAR comprising an antigen binding domain of claim 1, the nucleic acid encoding the binding protein or CAR, or a vector comprising the nucleic acid.

36. The cell of claim 35, wherein the cell is an immune effector cell and is a T cell, an NK cell, a CD8+ T cell and/or a human cell.

37. (canceled)

38. (canceled)

39. (canceled)

40. A composition comprising the nucleic acid of claim 30, a vector comprising the nucleic acid, or a cell comprising the nucleic acid or the vector a pharmaceutically acceptable carrier.

41. A method of making a CAR-expressing cell, comprising introducing the nucleic acid of claim 30 or a vector comprising the nucleic acid, into a cell, under conditions such that the CAR is expressed.

42. A method of treating a subject having a cancer associated with expression of EGFRvIII or a cancer associated with overexpression of EGFR, the method comprising administering to the subject the binding protein of claim 1, a composition thereof, or a cell comprising the binding protein or comprising a CAR comprising an antigen binding domain thereof.

43. The method of claim 42, wherein the cancer associated with expression of EGFRvIII is glioma, medulloblastoma, breast cancer, ovarian cancer, colon cancer, lung cancer, prostate cancer, pancreatic cancer, colorectal cancer, stomach cancer, renal cancer, cervical cancer, adenocarcinoma, bladder cancer, or head and neck cancer.

44. (canceled)

45. (canceled)

46. (canceled)

47. (canceled)

48. (canceled)

49. (canceled)

50. (canceled)

51. A method of detecting a cell expressing EGFRvIII, detecting a cell overexpressing EGFR, or diagnosing a subject with a cancer associated with expression of EGFRvIII or overexpression of EGFR, the method comprising
a) obtaining a sample comprising cells from the subject;
b) determining whether the cells express EGFRvIII or overexpress EGFR by contacting the sample with the binding protein of any one of claims 1 to 17 and detecting binding between the binding protein and EGFRvIII; and
c) detecting a cell expressing EGFRvIII or overexpressing EGFR or diagnosing the subject with the cancer associated with expression of EGFRvIII or overexpression of EGFR if binding is detected in step b).

52. (canceled)

53. (canceled)

54. A method of activating an immune effector cell comprising the CAR of claim 19, the method comprising contacting the immune effector cell with EGFRvIII.

55. The method of claim 54, further comprising administering the immune effector cell to the subject.

56. (canceled)

57. (canceled)


SEQUENCE LISTING (52 sequences)

SEQ ID NO: 1 - protein, 1210 aa, Homo sapiens [sequence omitted]

SEQ ID NO: 2 - protein, 942 aa, Homo sapiens
MRPSGTAGAALLALLAALCPASRALEEKKNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 3 - protein, 122 aa, Artificial Sequence
  Synthetic antibody sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEQWEGDYFSAFDIWGQGTMVTVSS

SEQ ID NO: 4 - protein, 110 aa, Artificial Sequence
  Synthetic antibody sequence
QSVLTQPPSVSVSPGQTASITCSGDKLGDKYACWYQQKPGQSPVLVIYQDSKRPSGIPERFSGSNSGNTATLTISGTQAMDEADYYCQAWDSDGKATVVFGTGTKVTVSS

SEQ ID NO: 5 - protein, 124 aa, Artificial Sequence
  Synthetic antibody sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGENTFESHPVAFDIWGQGTMVTVSS

SEQ ID NO: 6 - protein, 114 aa, Artificial Sequence
  Synthetic antibody sequence
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDLAADVRVVFGTGTKVTVSS

SEQ ID NO: 7 - protein, 8 aa, Artificial Sequence
  Synthetic antibody sequence
GFTFSSYA

SEQ ID NO: 8 - protein, 6 aa, Artificial Sequence
  Synthetic antibody sequence
SGSGGS

SEQ ID NO: 9 - protein, 15 aa, Artificial Sequence
  Synthetic antibody sequence
  X1 (1)..(1): Xaa is Glu or Gly
  X2 (2)..(2): Xaa is Gln or Gly
  X3 (3)..(3): Xaa is Trp or Glu
  X4 (4)..(4): Xaa is Glu or Asn
  X5 (5)..(5): Xaa is absent or is Thr
  X6 (6)..(6): Xaa is absent or is Phe
  X7 (7)..(7): Xaa is Gly or Glu
  X8 (8)..(8): Xaa is Asp or Ser
  X9 (9)..(9): Xaa is Tyr or His
  X10 (10)..(10): Xaa is Phe or Pro
  X11 (11)..(11): Xaa is Ser or Val
XXXXXXXXXXXAFDI

SEQ ID NO: 10 - protein, 9 aa, Artificial Sequence
  Synthetic antibody sequence
  X1 (1)..(1): Xaa is absent or is Ser
  X2 (2)..(2): Xaa is absent or is Ser
  X3 (3)..(3): Xaa is Asp or Gly
  X4 (4)..(4): Xaa is Lys or Ser
  X5 (5)..(5): Xaa is Leu or Ile
  X6 (6)..(6): Xaa is Gly or Ala
  X7 (7)..(7): Xaa is Asp or Ser
  X8 (8)..(8): Xaa is Lys or Asn
XXXXXXXXY

SEQ ID NO: 11 - protein, 7 aa, Artificial Sequence
  Synthetic antibody sequence
  X1 (1)..(1): Xaa is Gln or Glu
  X3 (3)..(3): Xaa is Ser or Asn
  X4 (4)..(4): Xaa is Lys or Gln
XDXXRPS

SEQ ID NO: 12 - protein, 12 aa, Artificial Sequence
  Synthetic antibody sequence
  X2 (2)..(2): Xaa is Ala or Ser
  X3 (3)..(3): Xaa is Trp or Tyr
  X5 (5)..(5): Xaa is Ser or Leu
  X6 (6)..(6): Xaa is Asp or Ala
  X7 (7)..(7): Xaa is Gly or Ala
  X8 (8)..(8): Xaa is Lys or Asp
  X9 (9)..(9): Xaa is Ala or Val
  X10 (10)..(10): Xaa is Thr or Arg
QXXDXXXXXXVV

SEQ ID NO: 13 - protein, 8 aa, Artificial Sequence
  Synthetic antibody sequence
GFTFSSYA

SEQ ID NO: 14 - protein, 6 aa, Artificial Sequence
  Synthetic antibody sequence
SGSGGS

SEQ ID NO: 15 - protein, 13 aa, Artificial Sequence
  Synthetic antibody sequence
EQWEGDYFSAFDI

SEQ ID NO: 16 - protein, 7 aa, Artificial Sequence
  Synthetic antibody sequence
DKLGDKY

SEQ ID NO: 17 - protein, 7 aa, Artificial Sequence
  Synthetic antibody sequence
QDSKRPS

SEQ ID NO: 18 - protein, 12 aa, Artificial Sequence
  Synthetic antibody sequence
QAWDSDGKATVV

SEQ ID NO: 19 - protein, 8 aa, Artificial Sequence
  Synthetic antibody sequence
GFTFSSYA

SEQ ID NO: 20 - protein, 6 aa, Artificial Sequence
  Synthetic antibody sequence
SGSGGS

SEQ ID NO: 21 - protein, 15 aa, Artificial Sequence
  Synthetic antibody sequence
GGENTFESHPVAFDI

SEQ ID NO: 22 - protein, 9 aa, Artificial Sequence
  Synthetic antibody sequence
SSGSIASNY

SEQ ID NO: 23 - protein, 7 aa, Artificial Sequence
  Synthetic antibody sequence
EDNQRPS

SEQ ID NO: 24 - protein, 12 aa, Artificial Sequence
  Synthetic antibody sequence
QSYDLAADVRVV

SEQ ID NO: 25 - protein, 518 aa, Artificial Sequence
  Synthetic CAR sequence
MDFQVQIFSFLLISASVIMSRGDGQSVLTQPPSVSVSPGQTASITCSGDKLGDKYACWYQQKPGQSPVLVIYQDSKRPSGIPERFSGSNSGNTATLTISGTQAMDEADYYCQAWDSDGKATVVFGTGTKVTVSSQTGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEQWEGDYFSAFDIWGQGTMVTVSSLSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 522 aa, Artificial Sequence
  Synthetic CAR sequence
MDFQVQIFSFLLISASVIMSRNFMLTQPHSVSESPGKTVTISCTRSSGSIASNYVQWYQQRPGSAPTTVIYEDNQRPSGVPDRFSGSIDSSSNSASLTISGLKTEDEADYYCQSYDLAADVRVVFGTGTKVTVSSQTGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGENTFESHPVAFDIWGQGTMVTVSSALSNSIMYFSHFVPVFLPAKPTTTPAPRPPTPAPTIASQPLSLRPEACRPAAGGAVHTRGLDPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 252 aa, Artificial Sequence
  Synthetic antibody sequence
QSVLTQPPSVSVSPGQTASITCSGDKLGDKYACWYQQKPGQSPVLVIYQDSKRPSGIPERFSGSNSGNTATLTISGTQAMDEADYYCQAWDSDGKATVVFGTGTKVTVSSQTGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEQWEGDYFSAFDIWGQGTMVTVSS

SEQ ID NO: 28 - protein, 258 aa, Artificial Sequence
  Synthetic antibody sequence
NFMLTQPHSVSESPGKTVTISCTRSSGSIASNYVQWYQQRPGSAPTTVIYEDNQRPSGVPDRFSGSIDSSSNSASLTISGLKTEDEADYYCQSYDLAADVRVVFGTGTKVTVSSQTGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGENTFESHPVAFDIWGQGTMVTVSS

SEQ ID NO: 29 - DNA, 366 bp, Artificial Sequence
  Synthetic antibody sequence
gaagtgcagctcctggagagcggtggcggactggtccagcccggaggatcgctgaggctctcatgcgccgcgtcaggattcaccttctcgtcgtacgccatgtcctgggtcagacaggcccctggaaagggactggaatgggtgtccgcgatctccggatcggggggctcaacttactacgccgactccgtgaagggccggttcaccatctcccgggataactccaagaacactctgtacttgcaaatgaactccctgcgcgccgaggataccgccgtgtactactgtgccaaggaacagtgggagggagattattttagcgcatttgacatctggggacagggaactatggtcaccgtcagctcc

SEQ ID NO: 30 - DNA, 333 bp, Artificial Sequence
  Synthetic antibody sequence
ggcgacggacagagcgtgctgactcagccaccctccgtgtccgtgagcccgggacagaccgcctccatcacgtgctccggggacaagctcggagacaaatacgcctgctggtaccaacagaagccgggacagagccctgtgcttgtgatctaccaggacagcaagcgcccgagcggcatccccgagcggttctccggctcgaactccgggaacactgccaccctgaccatttcgggaactcaggccatggacgaagcagactactactgccaagcgtgggattccgatgggaaagccaccgtggtgttcggaaccgggaccaaggtcaccgtg

SEQ ID NO: 31 - DNA, 372 bp, Artificial Sequence
  Synthetic sequence
gaagtgcagctcctggagagcggtggcggactggtccagcccggaggatcgctgaggctctcatgcgccgcgtcaggattcaccttctcgtcgtacgccatgtcctgggtcagacaggcccctggaaagggactggaatgggtgtccgcgatctccggatcggggggctcaacttactacgccgactccgtgaagggccggttcaccatctcccgggataactccaagaacactctgtacttgcaaatgaactccctgcgcgccgaggataccgccgtgtactactgtgccaagggaggggaaaacacatttgaatctcatccagtagcatttgacatctggggacagggaactatggtcaccgtcagctcc

SEQ ID NO: 32 - DNA, 336 bp, Artificial Sequence
  Synthetic antibody sequence
aacttcatgctgactcagccacattccgtgtccgaaagcccgggaaaaacagtgaccatctcctgcacgcgctcctccgggagcattgcgtccaactacgtgcagtggtaccaacagcggccggggtccgctcctaccaccgtgatctacgaggacaatcagcgcccgagcggcgtgcccgaccggttctccggctcgatcgactcgtcctcaaactccgccagcctgaccatttcgggacttaagactgaggacgaagcagactactactgccaaagctatgatctcgccgccgatgtccgcgtggtgttcggaaccgggaccaaggtcaccgtg

SEQ ID NO: 33 - DNA, 765 bp, Artificial Sequence
  Synthetic antibody sequence
ggcgacggacagagcgtgctgactcagccaccctccgtgtccgtgagcccgggacagaccgcctccatcacgtgctccggggacaagctcggagacaaatacgcctgctggtaccaacagaagccgggacagagccctgtgcttgtgatctaccaggacagcaagcgcccgagcggcatccccgagcggttctccggctcgaactccgggaacactgccaccctgaccatttcgggaactcaggccatggacgaagcagactactactgccaagcgtgggattccgatgggaaagccaccgtggtgttcggaaccgggaccaaggtcaccgtgtcatcccaaaccggcggatctggtggagggggcagcggtggtggaggctcaggcggtggaggcagtgaagtgcagctcctggagagcggtggcggactggtccagcccggaggatcgctgaggctctcatgcgccgcgtcaggattcaccttctcgtcgtacgccatgtcctgggtcagacaggcccctggaaagggactggaatgggtgtccgcgatctccggatcggggggctcaacttactacgccgactccgtgaagggccggttcaccatctcccgggataactccaagaacactctgtacttgcaaatgaactccctgcgcgccgaggataccgccgtgtactactgtgccaaggaacagtgggagggagattattttagcgcatttgacatctggggacagggaactatggtcaccgtcagctcc

SEQ ID NO: 34 - DNA, 774 bp, Artificial Sequence
  Synthetic sequence
aacttcatgctgactcagccacattccgtgtccgaaagcccgggaaaaacagtgaccatctcctgcacgcgctcctccgggagcattgcgtccaactacgtgcagtggtaccaacagcggccggggtccgctcctaccaccgtgatctacgaggacaatcagcgcccgagcggcgtgcccgaccggttctccggctcgatcgactcgtcctcaaactccgccagcctgaccatttcgggacttaagactgaggacgaagcagactactactgccaaagctatgatctcgccgccgatgtccgcgtggtgttcggaaccgggaccaaggtcaccgtgtcatcccaaaccggcggatctggtggagggggcagcggtggtggaggctcaggcggtggaggcagtgaagtgcagctcctggagagcggtggcggactggtccagcccggaggatcgctgaggctctcatgcgccgcgtcaggattcaccttctcgtcgtacgccatgtcctgggtcagacaggcccctggaaagggactggaatgggtgtccgcgatctccggatcggggggctcaacttactacgccgactccgtgaagggccggttcaccatctcccgggataactccaagaacactctgtacttgcaaatgaactccctgcgcgccgaggataccgccgtgtactactgtgccaagggaggggaaaacacatttgaatctcatccagtagcatttgacatctggggacagggaactatggtcaccgtcagctcc

SEQ ID NO: 35 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 39 aa, Homo sapiens
SKRSRLLHSDYMNMTPRRPGPTRKHYQPYAPPRDFAAYR

SEQ ID NO: 37 - protein, 30 aa, Homo sapiens
DPFWVLVVVGGVLACYSLLVTVAFIIFWVR

SEQ ID NO: 38 - protein, 58 aa, Homo sapiens
LSNSIMYFSHFVPVFLPAKPTTTPAPRPPTPAPTIASQPLSLRPEACRPAAGGAVHTR

SEQ ID NO: 39 - protein, 20 aa, Homo sapiens
DFQVQIFSFLLISASVIMSR

SEQ ID NO: 40 - protein, 404 aa, Artificial Sequence
  Synthetic protein construct
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRR

SEQ ID NO: 41 - protein, 5 aa, Artificial Sequence
  synthetic scFv sequence
SYAMS

SEQ ID NO: 42 - protein, 17 aa, Artificial Sequence
  synthetic scFv sequence
AISGSGGSTYYADSVKG

SEQ ID NO: 43 - protein, 9 aa, Artificial Sequence
  synthetic scFv sequence
EQWEGDYFS

SEQ ID NO: 44 - protein, 11 aa, Artificial Sequence
  synthetic scFv sequence
SGDKLGDKYAC

SEQ ID NO: 45 - protein, 7 aa, Artificial Sequence
  synthetic scFv sequence
QDSKRPS

SEQ ID NO: 46 - protein, 5 aa, Artificial Sequence
  synthetic scFv sequence
SDGKA

SEQ ID NO: 47 - protein, 5 aa, Artificial Sequence
  synthetic scFv sequence
SYAMS

SEQ ID NO: 48 - protein, 17 aa, Artificial Sequence
  synthetic scFv sequence
AISGSGGSTYYADSVKG

SEQ ID NO: 49 - protein, 11 aa, Artificial Sequence
  synthetic scFv sequence
GGENTFESHPV

SEQ ID NO: 50 - protein, 13 aa, Artificial Sequence
  synthetic scFv sequence
TRSSGSIASNYVQ

SEQ ID NO: 51 - protein, 7 aa, Artificial Sequence
  synthetic scFv sequence
EDNQRPS

SEQ ID NO: 52 - protein, 6 aa, Artificial Sequence
  synthetic scFv sequence
LAADVR